(19) United States
(12) Patent Application Publication
(10) Pub. No.: US 2023/0272357 A1
(43) Pub. Date: Aug. 31, 2023

(54) METHODS OF GENOME EDITING INCLUDING CONTROLLED OPENING OF CHROMATIN

(71) Applicant: Wisconsin Alumni Research Foundation, Madison, WI (US)

(72) Inventors: Krishanu Saha, Middleton, WI (US); Kaivalya Molugu, Watertown, MA (US)

(21) Appl. No.: 17/980,883

(22) Filed: Nov. 4, 2022

Related U.S. Application Data
(60) Provisional application No. 63/277,208, filed on Nov. 9, 2021.

(51) Int. Cl. C12N 9/22; C12N 15/10; A61P 43/00

(52) U.S. Cl. CPC C12N 9/22; C12N 15/102; A61P 43/00; C12N 2310/20

(57) ABSTRACT

Described herein is an ex vivo method of site-specifically editing a target cell genome, the method including treating a population of unmodified target cells with a Class I and/or Class II histone deacetylase inhibitor to provide a population of chromatin decondensed unmodified target cells; and introducing into the population of chromatin decondensed unmodified target cells a Cas9 ribonucleoprotein, to provide a population of site-specifically genome-edited target cells; wherein the Cas9 ribonucleoprotein comprises a Cas9 protein and a guide RNA and cleaves DNA at a cleavage site in the target cell genome.

### CROSS-REFERENCE TO RELATED APPLICATIONS

This application claims priority to U.S. Provisional Application 63/277,208 filed on Nov. 9, 2021, which is incorporated herein by reference in its entirety.

### SEQUENCE LISTING

The Instant Application contains a Sequence Listing which has been submitted electronically in XML format and is hereby incorporated by reference in its entirety. Said XML copy, created on Nov. 4, 2022, is named “WIS0065US2” and is 64,352 bytes in size.

### FIELD OF THE DISCLOSURE

The present disclosure is related to novel methods of editing in cells such as pluripotent stem cells, particularly methods that increase genome-editing efficiency.

### BACKGROUND

The pairing of gene editing technologies with human iPSCs for disease modeling overcomes the problem of animal models and human immortalized cell line models which do not accurately represent the genetic background or cellular physiology of the patient. Human iPSC-based models are thus a valuable resource for studying disease mechanisms, screening potential new therapeutics, and testing toxic side-effects of drug treatments. Moreover, performing gene editing on patient-derived iPSCs prior to differentiation enables the generation of isogenic iPSC lines which can function as a control for studying the genetic disease model of interest. Additionally, gene-edited iPSCs can be used for cellular therapies and could overcome the problem of immune rejection.

However, the process of gene-editing iPSCs is typically laborious and inefficient, involving multiple steps, requiring lengthy cell culture periods, drug selection, and several clonal events (i.e., gene targeting, and subsequent genetic excision of a selection cassette). The selection of rare-targeted clones in iPSCs can be particularly difficult because they grow poorly as single cells. Several recent approaches have been developed to improve the specificity, efficiency, and versatility of the CRISPR-Cas9 gene editing process by optimizing the structure of gRNAs, or by using modified nucleases strategies, natural and engineered Cas9 variants, and small molecules. However, these techniques still generate heterogeneous human cell populations that require time-consuming and laborious steps, significant subsequent characterization steps for on-target edits, off-target effects, genomic integrity, and unknown disease-causing mutations or risk variants.

Therefore, there is a need to overcome these limitations for enabling the use of gene-edited iPSC for cell therapies. Recent work indicates that chromatin structure of the target can have significant effects on Cas9 binding and gene editing efficiencies, thus leading to variation in targeting efficiency and choice of DNA repair pathway. Studies have shown that closed chromatin can negatively affect Cas9 binding or delay CRISPR-Cas9 mutagenesis, nucleosomes can block or present a hurdle for Cas9 access to DNA, and active transcription in open chromatin state can directly stimulate DNA cleavage by influencing Cas9 release states in a strand-specific manner Moreover, off-target binding of Cas9 to “seed” sequences has been shown to correlate with DNase I hypersensitivity sequences and inversely correlate with CpG methylation sites. Although these studies have shown chromatin structure can play a key role in gene editing and strategies have emerged to manipulate chromatin state to modulate gene editing efficiency, they are primarily based on immortalized cell lines or cancer cell lines which may not be clinically relevant. Moreover, though chromatin structure has been shown to impact off-target effects and the potency of iPSCs, the impact of chromatin structure on off-target effects and the potency of iPSCs in the context of gene editing has not been well characterized.

What is needed are novel strategies for genome-editing iPSCs and other cell types.

### BRIEF SUMMARY

In an aspect, an ex vivo method of site-specifically editing a target cell genome comprises treating a population of unmodified target cells with a Class I and/or Class II histone deacetylase inhibitor to provide a population of chromatin decondensed unmodified target cells; and introducing into the population of chromatin decondensed unmodified target cells a Cas9 ribonucleoprotein to provide a population of site-specifically genome-edited target cells; wherein the Cas9 ribonucleoprotein comprises a Cas9 protein and a guide RNA and cleaves DNA at a cleavage site in the target cell genome.

In another aspect, a method of allogenic or autologous cell therapy comprises transplanting differentiated genome-edited target cells into a subject in need thereof, wherein genome editing comprises the above-described method.

In another aspect, included herein are medical devices and in vitro disease models comprising differentiated genome-edited target cells, wherein genome editing comprises the above-described method.

In a further aspect, a method of guide-RNA design comprises treating a population of unmodified target cells with a Class I and/or Class II histone deacetylase inhibitor to provide a population of chromatin decondensed unmodified target cells; introducing into the population of chromatin decondensed unmodified target cells a Cas9 ribonucleoprotein to provide a population of genome-edited target cells, wherein the Cas9 ribonucleoprotein comprises a Cas9 protein and a first guide RNA, and cleaves DNA at a cleavage site in the target cell genome; determining the percentage of off-target genome edited sites in the population of genome-edited target cells compared to on-target genome-edited sites; and designing a second guide RNA when the percentage of off-target genome edited sites produced by the first guide RNA is greater than half of the percentage of the on-target genome edits produced by the first guide RNA, wherein the second guide RNA is predicted to reduce off-target genome-edited sited compared to the first guide RNA.

### BRIEF DESCRIPTION OF THE DRAWINGS

FIGS. 1A-H show trichostatin A (TSA) increases CRISPR-Cas9 mediated gene editing efficiency of iPSCs at HIST1H2BJ-GFP locus. 1A) Schematic showing the TSA-based gene editing strategy and analyses. 1B) Schematic of the HIST1H2BJ-GFP locus with gRNA target sequence, PAM sequence, and cut-site (black arrow) labelled. The locus strands are: SEQ ID NO: 1 (CCTGGCCCACCCTCGTGACCACCCTGACCTACGGCGTGCAGTGCTTCAGCCGCTA CCCCGACCACA), SEQ ID NO:2 (TGTGGTCGGGGTAGCGGCTGAAGCACGCCGTAGGTCAGGGTGGTCACGAGGGT GGGCCAGG). 1C) Representative images of HIST1H2BJ-GFP reporter iPSC nuclei after TSA treatment (0, 3.13, 6.25, 12.5 ng/mL). Bright green spots indicate heterochromatin foci; Scale bar: 10 μm. 1D) Box plot showing the distribution of chromatin condensation %. Chromatin condensation % decreases with the application of TSA (n=20 nuclei, 3 technical replicates per condition). The quantification bar graphs indicate that cell viability (% GhostDye™-cells/total cells) decreases, transfection efficiency (% ATTO 550+cells/viable cells) does not change significantly, and gene editing efficiency (% GFP−cells/viable cells) increases upon TSA treatment. Data represented in bar graphs are represented as mean±SEM, n=6 technical replicates per condition from 2 independent experiments, p-values generated by Mann-Whitney non-parametric t-test for multiple comparisons to 0 ng/mL TSA; ns=p>0.05, * for p<0.05, ** for p<0.01, *** for p<0.001, **** for p<0.0001. 1E) Representative density flow cytometry plots showing Ghost Dye™ Red 780 viability dye levels on iPSCs treated with TSA. 1F) Histograms showing ATTO 550 expression on day 2 after Cas9 RNP delivery. 1H) Histograms showing GFP expression on day 6 after Cas9 RNP delivery.

FIGS. 2A-F show TSA increases gene editing efficiency at seven open and closed chromatin loci in iPSCs, and results for deep sequencing which reveal a shift in indel profiles upon TSA treatment. 2A) Bar graphs showing increased % editing efficiencies upon TSA treatment at open loci: HIST1H2BJ-GFP, AAVS1 (two sites: S8, S10); and closed loci: EMX1, VEGFA, and TRAC (two sites: S2, S3) loci. 2B) Bar graphs showing no significant change in % edited reads with insertions upon TSA treatment. 2C) Representative insertion profiles 20 bp around the gRNA cut site for all the seven loci. 2D) Bar graph showing increased % edited reads with deletions upon TSA treatment. 2E) Representative deletion profiles 20 bp around the gRNA cut site for all the seven loci. 2F) Bar graphs showing decreased % edited reads with only SNPs upon TSA treatment at all seven loci. Data represented in bar graphs are represented as mean±SEM, n=3 technical replicates per condition, p-values generated by two-way ANOVA Dunnett's multiple comparison test for multiple comparisons to 0 ng/mL TSA; ns=p>0.05, * for p<0.05, ** for p<0.01, *** for p<0.001, **** for p<0.0001.

FIGS. 3A-E show off-target analysis of edited iPSCs. 3A) Visualization of SpCas9 target and off-target sites detected by CHANGE-seq for the HIST1H2BJ-GFP gRNA. The intended target GFP sequence (SEQ ID NO: 3; GCTGAAGCACTGCACGCCGTNGG) is shown in the top line. Cleaved sites (off-target) are shown below and are ordered top to bottom by CHANGE-seq read count, with mismatches to the intended target sequence indicated by colored nucleotides. Insertions are shown in smaller lettering between genomic positions, deletions are shown by (−). Note that output is truncated to the top 12 sites. 3B) Manhattan plot of CHANGE-seq-detected off-target sites organized by chromosomal position with bar heights representing CHANGE-seq read count. 3C-E) Left: Indel % at 3C) HIST1H2BJ-GFP, 3B) AAVS1 S10, 3E) EMX1 on-target and top off-target sites detected by CHANGE-seq or GUIDE-seq, assayed by rhAmpSeq system. While the on-target indel % increases with TSA concentration, the off-target indel % remains the same. Data represented in bar graphs are represented as mean±SEM, n=3 technical replicates per condition, p-values generated by two-way ANOVA Dunnett's multiple comparison test for multiple comparisons to 0 ng/mL TSA; ns=p>0.05, * for p<0.05, ** for p<0.01, *** for p<0.001, **** for p<0.0001. Right: Normalized on-target edit ratio for each of the top off-target sites at 3C) HIST1H2BJ-GFP, 3B) AAVS1 S10, 3E) EMX1 plotted as a function of TSA concentration. TSA concentration of 6.25 ng/mL yields the highest normalized on-target edit ratio.

FIGS. 4A-E show karyotypic analysis of edited iPSC lines. Four of the five edited isolated iPSC clones (FIG. 4A-D; TSA treatment; 6.25 ng/mL) showed normal karyotype indicating that cell lines with no major chromosome abnormalities can be isolated after TSA-induced gene editing. One clone #5 (FIG. 4E) showed an interstitial duplication in the long (q) arm of chromosome 20 in five of the twenty cells examined

### DETAILED DESCRIPTION

Described herein is a strategy to modify the impact of chromatin structure on gene editing outcomes in cells such as iPSCs. In an aspect, gene editing is via the application of a small molecule known to promote open chromatin state, called Trichostatin A (TSA; Class I and II histone deacetylase inhibitor or HDAC inhibitor). First, live, in situ nuclear imaging was used to quantify the TSA-induced change in global chromatin state of iPSC nuclei, called chromatin condensation. Second, extensive characterization was performed to assess the impact of TSA on gene editing outcomes, i.e., on-target efficiencies, off-target effects, pluripotency, and genomic integrity of the iPSCs. Chromatin decondensation via TSA increased the gene editing efficiency of iPSCs by approximately 2-3 fold, while simultaneously ensuring genomic integrity and no increased off-target effects. Overall, the methods described herein provide a strategy for rapid and in situ imaging-based identification of iPSCs amenable to gene editing, and also engineered the gene editing process to generate edited cells such as iPSCs efficiently.

In an aspect, an ex vivo method of site-specifically editing a target cell genome comprises treating a population of unmodified target cells with a Class I and/or Class II histone deacetylase inhibitor to provide a population of chromatin decondensed unmodified target cells; and introducing into the population of chromatin decondensed unmodified target cells a Cas9 ribonucleoprotein, to provide a population of site-specifically genome-edited target cells; wherein the Cas9 ribonucleoprotein comprises a Cas9 protein and a guide RNA and cleaves DNA, e.g., double-stranded DNA, at a cleavage site in the target cell genome.

In the methods described herein, a Class I and/or Class II histone deacetylase inhibitor provides a population of chromatin decondensed unmodified target cells.

Exemplary Class I and/or Class II histone deacetylase inhibitors include vorinostat, panobinostat, belinostat, entinostat, phenyl butyrate, valproic acid, trichostatin A, mocetinostat, pracinostat, dacinostat, givinostat, abexinostat, depsipeptide, and combinations thereof, specifically trichostatin A. When the Class I and/or Class II histone deacetylase inhibitor is trichostatin A, treating a population of unmodified target cells with the Class I and/or Class II histone deacetylase inhibitor may be done for 16 to 24 hours at a temperature of 37° C., and a concentration of 3.125 ng/mL to 12.5 ng/mL.

Advantageously, the methods described herein decondense chromatin and thus result in dramatically improved editing efficiencies of genome editing using an Cas9 RNP. In contrast to prior methods that have utilized delivery of plasmids encoding Cas9 and the guide RNAs, the methods described herein comprising RNP delivery result in lower lifetimes of nuclease within the cell. Therefore, the timing of decondensing chromatin may be different than in prior studies that have manipulated chromatin to augment genome editing. The methods described herein may also modify the on-target to off-target ratio differently relative to delivery strategies employing plasmids, mRNAs and viral vectors. The method can further comprise live in situ nuclear imaging of the population of chromatin decondensed unmodified target cells, and quantifying the histone deacetylase inhibitor-induced decrease in chromatin condensation of the target cell nuclei. In an aspect, the histone deacetylase inhibitor-induced decrease in chromatin condensation percentage is at least a 1% decrease in chromatin condensation percentage calculated as a percentage of heterochromatin intensity to the total nuclear intensity.

Once the chromatin is decondensed, a Cas9 RNP is used to provide a population of genome-edited target cells. As used herein a Cas9 RNP comprises a Cas9 protein and a guide RNA that directs DNA cleavage of a cleavage site in a target cell expressed gene.

As used herein a guide RNA can be a two-part guide RNA comprising a CRISPR RNA (crRNA) that includes a 20 base protospacer element that is complementary to a genomic DNA sequence as well as additional elements that are complementary to the transactivating RNA (tracrRNA). The tracrRNA hybridizes to the crRNA and binds to the Cas9 protein, to provide an active RNP complex. In another aspect, the guide RNA can be an sgRNA refers to a single RNA species which combines the tracrRNA and the crRNA and is capable of directing Cas9-mediated cleavage of target DNA. An sgRNA thus contains the sequences necessary for Cas9 binding and nuclease activity and a target sequence complementary to a target DNA of interest (protospacer sequence). In general, in an sgRNA, the tracrRNA and the crRNA are connected by a linker loop sequence. sgRNAs are well-known in the art.

Any desired target DNA sequence of interest may be targeted by a guide RNA target sequence. Any length of target sequence that permits CRISPR-Cas9 specific nuclease activity may be used in a guide RNA. In some embodiments, a guide RNA contains a 20 nucleotide protospacer sequence.

In addition to the protospacer sequence, the targeted sequence includes a protospacer adjacent motif (PAM) adjacent to the protospacer region which is a sequence recognized by the CRISPR RNP as a cutting site. Without wishing to be bound to theory, it is thought that the only requirement for a target DNA sequence is the presence of a protospacer-adjacent motif (PAM) adjacent to the sequence complementary to the guide RNA target sequence. Different Cas9 complexes are known to have different PAM motifs. For example, Cas9 from Streptococcus pyogenes has a NGG trinucleotide PAM motif; the PAM motif of N. meningitidis Cas9 is NNNNGATT; the PAM motif of S. thermophilus Cas9 is NNAGAAW; and the PAM motif of T. denticola Cas9 is NAAAAC.

A “Cas9” protein is a polypeptide that functions as a nuclease when complexed to a guide RNA, e.g., an sgRNA or modified sgRNA. The Cas9 (CRISPR-associated 9, also known as Csn1) family of polypeptides, for example, when bound to a crRNA:tracrRNA guide or single guide RNA, are able to cleave target DNA at a sequence complementary to the sgRNA target sequence and adjacent to a PAM motif as described above. Cas9 polypeptides are characteristic of type II CRISPR-Cas systems. The broad term “Cas9” Cas9 polypeptides include natural sequences as well as engineered Cas9 functioning polypeptides. The term “Cas9 polypeptide” also includes the analogous Clustered Regularly Interspaced Short Palindromic Repeats from Prevotella and Francisella 1 or CRISPR/Cpf1 which is a DNA-editing technology analogous to the CRISPR/Cas9 system. Cpf1 is an RNA-guided endonuclease of a class II CRISPR/Cas system. This acquired immune mechanism is found in Prevotella and Francisella bacteria. Additional Class I Cas proteins include Cas3, Cas8a, Cas5, Cas8b, Cas8c, Cas 10d, Case1, Cse 2, Csy 1, Csy 2, Csy 3, GSU0054, Cas 10, Csm 2, Cmr 5, Cas10, Csx11, Csx10, and Csf 1. Additional Class 2 Cas9 polypeptides include Csn 2, Cas4, C2c1, C2c3 and Cas13a.

Exemplary Cas9 polypeptides include Cas9 polypeptide derived from Streptococcus pyogenes, e.g., a polypeptide having the sequence of the Swiss-Prot accession Q99ZW2 (SEQ ID NO: 52); Cas9 polypeptide derived from Streptococcus thermophilus, e.g., a polypeptide having the sequence of the Swiss-Prot accession G3ECR1 (SEQ ID NO: 53); a Cas9 polypeptide derived from a bacterial species within the genus Streptococcus; a Cas9 polypeptide derived from a bacterial species in the genus Neisseria (e.g., GenBank accession number YP_003082577; WP_015815286.1 (SEQ ID NO: 54)); a Cas9 polypeptide derived from a bacterial species within the genus Treponema (e.g., GenBank accession number EMB41078 (SEQ ID NO: 55)); and a polypeptide with Cas9 activity derived from a bacterial or archaeal species. Methods of identifying a Cas9 protein are known in the art. For example, a putative Cas9 protein may be complexed with crRNA and tracrRNA or sgRNA and incubated with DNA bearing a target DNA sequence and a PAM motif.

The term “Cas9” or “Cas9 nuclease” refers to an RNA-guided nuclease comprising a Cas9 protein, or a fragment thereof (e.g., a protein comprising an active, inactive, or partially active DNA cleavage domain of Cas9, and/or the gRNA binding domain of Cas9). In some embodiments, a Cas9 nuclease has an inactive (e.g., an inactivated) DNA cleavage domain, that is, the Cas9 is a nickase. Other embodiments of Cas9, both DNA cleavage domains are inactivated. This is referred to as catalytically-inactive Cas9, dead Cas9, or dCas9.

Functional Cas9 mutants are described, for example, in US20170081650 and US20170152508, incorporated herein by reference for its disclosure of Cas9 mutants.

The term Cas9 RNP also included modified Cas9 RNPs. U.S. Pat. No. 10,907,150, incorporated by reference, describes modified RNP complexes, referred to as Simplexes, which include a guide RNA comprising a nucleic acid aptamer that binds an avidin. When the complex also includes an avidin, a biotinylated donor DNA will associate with the complex, reducing errors typically associated with CRISPR gene editing.

Modified Cas9 RNPs also include RNPs comprising conjugated moieties such as cell-penetrating peptides, antibodies, nucleic acids, cell targeting peptides/molecules. The conjugated moieties can provide cell targeting, for example, to edit cells selectively.

The Cas9 RNP directs DNA cleavage of a cleavage site in the target cell genome. In an aspect, the cleavage site in the target cell genome creates a precise knockout in the target cell genome, such as a knockout of a target cell expressed gene. Advantageously, genome editing proceeds primarily by a non-homologous end-joining repair pathway (NHEJ). In addition, by using the methods described herein, the percentage of off-target genome edited sites is less than half of the percentage of the on-target genome edits.

In another aspect, the method further comprises introducing into the population of chromatin decondensed unmodified target cells a donor polynucleotide comprising a synthetic DNA sequence flanked by homology arms that are complementary to sequences on both sides of the cleavage site in the target cell genome, wherein the synthetic DNA sequence in the donor polynucleotide is specifically integrated into the cleavage site of the target cell genome.

Homologous recombination can insert an exogenous polynucleotide sequence into the target nucleic acid cleavage site. An exogenous polynucleotide sequence can be called a donor polynucleotide or a donor sequence. In some embodiments, a donor polynucleotide, a portion of a donor polynucleotide, a copy of a donor polynucleotide, or a portion of a copy of a donor polynucleotide can be inserted into a target nucleic acid cleavage site. A donor polynucleotide can be single-stranded DNA, double-stranded DNA, RNA, or a duplex of RNA and DNA. A donor polynucleotide can be a sequence that does not naturally occur at a target nucleic acid cleavage site. In some embodiments, modifications of a target nucleic acid due to NHEJ and/or HDR can lead to, for example, mutations, deletions, alterations, integrations, gene correction, gene replacement, transgene insertion, nucleotide deletion, gene disruption, and/or gene mutation. NHEJ repair of Cas9 RNP cleavages is useful to make knockouts of a gene of interest. In this case, the break ends are ligated in the absence of a donor polynucleotide. HDR, in contrast, requires a donor polynucleotide to guide repair. The process of integrating non-native nucleic acid(s) into genomic DNA can be referred to as “genome engineering”.

In specific aspects, the donor polynucleotide includes a mutation, deletion, alteration, integration, gene correction, gene replacement, transgene insertion, nucleotide deletion, gene disruption, and/or gene mutation.

Exemplary cleavage sites in the target cell genome include a target cell expressed gene, a regulatory region, noncoding RNA, repeat region, integrated viral genome, topologically associating domain, and lamina-associated domain. Exemplary regulatory regions include enhancers, promoters, repressors, silencers, insulators, and the like.

Exemplary target cells for the genome editing methods described herein include induced pluripotent stem cells (iPSCs), hematopoietic stem cells (HPSCs), neural progenitor cells, natural killer cells (NK cells), and T cells, specifically, iPSCs or HPSCs.

As used herein, the term “induced pluripotent stem cells” or, iPSCs, means that the stem cells are produced from differentiated adult, neonatal or fetal cells that have been induced or changed, i.e., reprogrammed into cells capable of differentiating into tissues of all three germ or dermal layers: mesoderm, endoderm, and ectoderm. The iPSCs produced do not refer to cells as they are found in nature.

Pluripotency can be determined, in part, by assessing pluripotency characteristics of the cells. Pluripotency characteristics include, but are not limited to: (i) pluripotent stem cell morphology; (ii) the potential for unlimited self-renewal; (iii) expression of pluripotent stem cell markers including, but not limited to SSEA1 (mouse only), SSEA3/4, SSEAS, TRA1-60/81, TRA1-85, TRA2-54, GCTM-2, TG343, TG30, CD9, CD29, CD133/prominin, CD140a, CD56, CD73, CD90, CD105, OCT4, NANOG, SOX2, CD30 and/or CD50; (iv) ability to differentiate to all three somatic lineages (ectoderm, mesoderm and endoderm); (v) teratoma formation consisting of the three somatic lineages; and (vi) formation of embryoid bodies consisting of cells from the three somatic lineages.

As used herein, the term “embryonic stem cell” refers to naturally occurring pluripotent stem cells of the inner cell mass of the embryonic blastocyst. Embryonic stem cells are pluripotent and give rise during development to all derivatives of the three primary germ layers: ectoderm, endoderm and mesoderm. They do not contribute to the extra-embryonic membranes or the placenta, i.e., are not totipotent.

The term “hematopoietic stem cells” or HSPCs refers to cells which are committed to a hematopoietic lineage but are capable of further hematopoietic differentiation and include, multipotent hematopoietic stem cells (hematoblasts), myeloid progenitors, megakaryocyte progenitors, erythrocyte progenitors, and lymphoid progenitors. Hematopoietic stem and progenitor cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T cells, B cells, NK cells). The term “definitive hematopoietic stem cell” as used herein, refers to CD34+ hematopoietic cells capable of giving rise to both mature myeloid and lymphoid cell types including T cells, NK cells and B cells. Hematopoietic cells also include various subsets of primitive hematopoietic cells that give rise to primitive erythrocytes, megakarocytes and macrophages.

As used herein, “neural progenitor cells” are the progenitor cells of the central nervous system that give rise to glial and neuronal cell types. Unlike neural stem cells, neural progenitor cells have limited proliferative ability.

As used herein, the terms “T lymphocyte” and “T cell” are used interchangeably and refer to a principal type of white blood cell that completes maturation in the thymus and that has various roles in the immune system, including the identification of specific foreign antigens in the body and the activation and deactivation of other immune cells. A T cell can be any T cell, such as a cultured T cell, e.g., a primary T cell, or a T cell from a cultured T cell line, e.g., Jurkat, SupT1, etc., or a T cell obtained from a mammal. The T cell can be CD3⁺ cells. The T cell can be any type of T cell and can be of any developmental stage, including but not limited to, CD4⁺/CD8⁺ double positive T cells, CD4⁺ helper T cells (e.g., Th1 and Th2 cells), CD8⁺ T cells (e.g., cytotoxic T cells), peripheral blood mononuclear cells (PBMCs), peripheral blood leukocytes (PBLs), tumor infiltrating lymphocytes (TILs), memory T cells, naive T cells, regulator T cells, gamma delta T cells (γδT cells), and the like. Additional types of helper T cells include cells such as Th3 (T_{reg}), Th17, Th9, or T_{fh} cells. Additional types of memory T cells include cells such as central memory T cells (Tₑₘ cells), effector memory T cells (Tₑₘ cells and TEMRA cells). The T cell can also refer to a genetically engineered T cell, such as a T cell modified to express a T cell receptor (TCR) or a chimeric antigen receptor (CAR). The T cell can also be differentiated from a stem cell or progenitor cell.

“CD4⁺ T cells” refers to a subset of T cells that express CD4 on their surface and are associated with cell-mediated immune response. They are characterized by the secretion profiles following stimulation, which may include secretion of cytokines such as IFN-gamma, TNF-alpha, IL2, IL4 and IL10. “CD4” are 55-kD glycoproteins originally defined as differentiation antigens on T-lymphocytes, but also found on other cells including monocytes/macrophages. CD4 antigens are members of the immunoglobulin supergene family and are implicated as associative recognition elements in MHC (major histocompatibility complex) class II-restricted immune responses. On T-lymphocytes they define the helper/inducer subset.

“CD8⁺ T cells” refers to a subset of T cells which express CD8 on their surface, are MHC class I-restricted, and function as cytotoxic T cells. “CD8” molecules are differentiation antigens found on thymocytes and on cytotoxic and suppressor T-lymphocytes. CD8 antigens are members of the immunoglobulin supergene family and are associative recognition elements in major histocompatibility complex class I-restricted interactions.

As used herein, the term “NK cell” or “Natural Killer cell” refer to a subset of peripheral blood lymphocytes defined by the expression of CD56 or CD16 and the absence of the T cell receptor (CD3). As used herein, the terms “adaptive NK cell” and “memory NK cell” are interchangeable and refer to a subset of NK cells that are phenotypically CD3⁻ and CD56⁺, expressing at least one of NKG2C and CD57, and optionally, CD16, but lack expression of one or more of the following: PLZF, SYK, FceR gamma., and EAT-2. In some embodiments, isolated subpopulations of CD56⁺ NK cells comprise expression of CD16, NKG2C, CD57, NKG2D, NCR ligands, NKp30, NKp40, NKp46, activating and inhibitory KIRs, NKG2A and/or DNAM-1. CD56⁺ can be dim or bright expression.

In an aspect, the method can further comprise selecting a clone from the population of genome-edited target cells and expanding the clone to provide a population of clonally expanded genome-edited cells. Culture plates and/or bioreactors using media known in the art can be used to expand the selected clones.

In a specific aspect, the gene expression of pluripotency genes OCT4, NANOG, SOX2, and TRA-1-60 is decreased by less than 50% in the clonally expanded genome-edited cells compared to clonally expanded genome-edited cells produced in the absence of the II histone deacetylase inhibitor.

The expanded cell population can then be differentiated to provide differentiated genome-edited cells. For example, iPSCs can be differentiated to provide alveolar epithelial cells, airway epithelial cells, neuronal cells, adipocytes, cardiomyocytes, hematopoietic cells, pancreatic beta cells, retinal epithelial cells, photoreceptors, retinal ganglion cells, epidermal cells, intestinal epithelial cells, smooth muscle cells, skeletal muscle cells, renal cells, chondrocytes, osteocytes, stromal cells, T cells, natural killer cells, macrophages red blood cells, and the like.

The differentiated genome -edited cells produced by the methods described herein are particularly useful in methods of allogenic or autologous cell/gene therapy. In allogenic therapy, the unmodified target cells are sourced from the same person who will receive the transplant. In allogenic cell therapy, the unmodified target cells are from a matched related or unrelated donor. In an aspect, the differentiated genome -edited cells are in the form of a transfusion, a tissue transplant, or a medical device. Transfusion, for example, can be used for HPSC delivery. Tissue transplant can be used for bone marrow transplant. Medical devices can include scaffolds, supports, gels, pastes and the like which can be used as a support for implantation of the differentiated genome -edited cells. Such medical devices can be seeded with mesenchymal stem cells for orthopedic indications such as knee, hip, shoulder, spine, elbow, hand, wrist, foot and ankle injuries. Devices also include insulin producing beta cells derived from iPSCs to treat diabetes, and retinal tissues generated from iPSCs to restore vision.

A high dose of monoclonal antibodies, chemotherapy and/or radiation therapy may precede allogenic or autologous cell/gene therapy. This therapy is called “conditioning treatment” and helps make room for the transplanted cells to grow, helps prevent rejection of the transplanted cells, and/or helps kill any cancer cells in the body.

Allogenic or autologous cell/gene therapy can be used to treat inherited cardiac disease, Huntington's disease, Alzheimer's disease, Parkinson's disease, schizophrenia, amyotrophic lateral sclerosis, spinal muscular atrophy, Rett syndrome, Prader-Willi syndrome, non-Hodgkin lymphoma, Hodgkin lymphoma, leukemia, multiple myeloma, aplastic anemia, diabetes, sickle cell disease, thalassemia, lysosomal storage diseases, Duchenne's Muscular Dystrophy, inherited retinal disorder, or cystic fibrosis, cancer, kidney disease, a liver disease, and the like.

Also included herein are in vitro disease models comprising the genome-edited target cells described herein. In vitro disease models are particularly useful to study the causes, pathologies and mechanisms of diseases, as well as to model disease outcome, screen a target drug, biologic or genetic medicine treatment, or test toxic side-effects of a treatment.

In another aspect, a method of guide-RNA design comprises treating a population of unmodified target cells with a Class I and/or Class II histone deacetylase inhibitor to provide a population of chromatin decondensed unmodified target cells; introducing into the population of chromatin decondensed unmodified target cells a Cas9 ribonucleoprotein, to provide a population of genome-edited target cells, wherein the Cas9 ribonucleoprotein comprises a Cas9 protein and a first guide RNA, and cleaves DNA at a cleavage site in the target cell genome; determining the percentage of off-target genome edited sites in the population of genome-edited target cells compared to on-target genome-edited sites; and designing a second guide RNA when the percentage of off-target genome edited sites produced by the first guide RNA is greater than half of the percentage of the on-target genome edits produced by the first guide RNA, wherein the second guide RNA is predicted to reduce off-target genome-edited sited compared to the first guide RNA.

The foregoing method can be used iteratively, for example, to iteratively provide improved guide RNAs. In an aspect, the method further comprises designing one or more subsequent guide RNAs to the second guide RNAs, wherein the designing optimizes the percentage of off-target genome edited sites in the population of genome-edited target cells compared to on-target genome-edited sites.

The above-described and other features will be appreciated and understood by those skilled in the art from the following detailed description, drawings, and appended claims.

The invention is further illustrated by the following non-limiting examples.

### EXAMPLES

### Methods

Cell culture: Mono-allelic mEGFP-tagged HIST1H2BJ human iPSCs (AICS-0061-036) were obtained from Allen Institute for Cell Science. This cell line was derived from the WTC parental line (GM25256) released by the Conklin Laboratory at the J. David Gladstone Institute. iPSCs were maintained in mTeSR1 medium on Matrigel® (WiCell) coated tissue culture polystyrene plates (BD Falcon). Cells were passaged every 4-5 days at a ratio of 1:8 using ReLeSR™ solution (STEMCELL Technologies). All cells were maintained at 37° C. in 5% CO₂ and tested monthly for possible mycoplasma contamination.

Chemical reagents: Trichostatin A (Millipore Sigma) was resuspended in DMSO at stock concentrations of 2 mg/mL. Aliquots were then stored at −20° C. The purity of the inhibitor was assessed by Millipore Sigma (>99%).

Cell viability: Flow cytometry was performed using Ghost Dye™ Red 780 viability dye (Tonbo Biosciences) to determine the dose range of the TSA that can be administrated to the cells without affecting cell viability. iPSCs (15,000 per well) were cultured overnight and incubated with TSA in 96-well plates for 24 h. The next day, cells were singularized using Accutase™ (STEMCELL technologies), washed with PBS, centrifuged at 300 g for 5 minutes, and Ghost Dye™ Red 780 viability dye was added at 1:1000 concentration for 20 minutes at room temperature. Cells were then washed with PBS, spun down at 300g for 5 minutes, and resuspended in 300 μL of PBS. Cells were run on an Attune NxT™ flow cytometer (Thermo Fisher Scientific) and subsequent analysis was performed using Flowjo software (BD).

SpyCas9 RNP preparation: RNPs were produced by complexing a two-component gRNA to SpyCas9. In brief, tracr-RNA and crRNA were ordered from IDT, suspended in nuclease-free duplex buffer at 100 μM, and stored in single-use aliquots at −20° C. tracrRNA and crRNA were thawed, and 0.0625 μL of each component was mixed 1:1 by volume and annealed by incubation at room temperature for 5 minutes to form a 50 μM sgRNA solution for each well of a 96 well plate. Recombinant sNLS-SpCas9-sNLS Cas9 (Aldevron, 10 mg/mL) was added to the complexed gRNA at a 1:1 molar ratio (1000 ng/well, Total 0.1 μL) and incubated for 5 minutes at room temperature to form RNP.

RNP delivery: iPSCs were singularized using Accutase™ and counted using a Countess® II FL Automated Cell Counter (Thermo Fisher Scientific) with 0.4% Trypan Blue viability stain (Thermo Fisher Scientific). iPSCs were then seeded at 15,000 cells/well on 96 well glass bottom plates (Cellvis) in mTeSR1 (WiCell) and 10 μM ROCK inhibitor (Y27632, Selleckchem), two days before lipofection or electroporation. On the following day, iPSCs were treated with TSA (0-200 ng/mL) for 16-24 hours. iPSC lipofections were performed using 0.5 μL Lipofectamine Stem Cell Reagent/well (1000 ng Cas9/well and sgRNA at 1:1 molar ratio). Cells remained undisturbed for 48 hours and were then passaged 1:4 using ReLeSR™ solution followed by daily mTeSR1 media changes for 4 additional days before downstream analysis.

iPSC electroporations were performed using the 4D-Nucleofector™ System (Lonza) as per the manufacturer's instructions. Briefly, iPSCs were harvested using Accutase™ (STEMCELL Technologies) and counted. 2×105 cells per electroporation were then centrifuged at 300 g for 5 min. Media was aspirated and cells were resuspended using 20 μl of P3 solution (Lonza) with 3μg of Cas9 and sgRNA at a 1:1 molar ratio. iPSCs were then electroporated using protocol CB-150. After nucleofection, samples were incubated in nucleocuvettes at room temperature for 15 min before plating into 6×10⁴ cells per well on 96 well glass bottom plate in mTeSR1 media+10 μM ROCK inhibitor. Media was changed 24 hours post-transfection and replaced with mTeSR1 medium.

Flow cytometry and fluorescence activated cell sorting: Flow cytometry was performed on singularized iPSCs using Attune™ Nxt flow cytometer (ThermoFisher Scientific) and analyzed using the FlowJo Software. Ghost Dye™ Red 780, ATTO 550, and GFP fluorescence were detected using 780/60, 585/16, and 530/30 filters in BL1, YL1, and RL3 positions, respectively. Gates were established by running singularized untransfected iPSCs. The percentage of viable cells was calculated as the ratio of Ghost Dye™ Red 780-cells to the total number of single cells. Transfection efficiency was calculated as the percentage of ATTO 550+ cells to the total number of viable cells on day 2 post-transfection. Gene editing efficiency was calculated as the percentage of GFP-cells to the total number of viable cells on day 6 post-transfection. For the sorting experiments, iPSCs were singularized 6 days post-transfection using Accutase™, washed with PBS+20 μM ROCK inhibitor, centrifuged at 300 g for 5 minutes. Ghost Dye™ Red 780 viability dye (Tonbo Biosciences) was then added at 1:1000 concentration for 20 minutes at room temperature. Cells were washed again with PBS+20 μM ROCK inhibitor, spun down at 300 g for 5 minutes, and resuspended in 300 μL of FACS buffer (PBS+2% BSA+20 μM ROCK inhibitor). GFP-cells were then sorted into tubes containing mTeSR1+20 μM ROCK inhibitor, and seeded onto Matrigel®-coated 6 well polystyrene plates at 100 cells/well to obtain single-cell iPSC clones.

Next Generation Sequencing of genomic DNA: DNA was isolated from iPSCs by adding 50 μL of DNA QuickExtract™/well (Epicentre) following treatment by Accutase™ and centrifugation. The DNA extract solution was incubated at 65° C. for 15 min, 68° C. for 15 min, and finally 98° C. for 10 min. Genomic PCR was performed according to the manufacturer's instructions using Q5 Hot Start polymerase (NEB). Sequencing indices were added with a second round of PCR using indexing primers (IDT), followed by a purification using AMPure XP magnetic bead purification kit (Beckman Coulter). Samples were pooled and sequenced on an Illumina® MiniSeq at a run length of 1×150 bp or 2×150 bp according to the manufacturer's instructions. Analysis was performed using CRISPR RGEN.

Genome-wide, off-target analysis: Genomic DNA from iPSCs was isolated using Gentra® Puregene® Kit (Qiagen) according to the manufacturer's instructions. CHANGE-seq was performed as described in the art. Briefly, purified genomic DNA was tagmented with a custom Tn5-transposome to an average length of 400 bp, followed by gap repair with Kapa HiFi HotStart Uracil+DNA Polymerase (KAPA Biosystems) and Taq DNA ligase (NEB). Gap-repaired tagmented DNA was treated with USER™ enzyme (NEB) and T4 polynucleotide kinase (NEB). Intramolecular circularization of the DNA was performed with T4 DNA ligase (NEB) and residual linear DNA was degraded by a cocktail of exonucleases containing Plasmid-Safe ATP-dependent DNase (Lucigen), Lambda exonuclease (NEB), and Exonuclease I (NEB). In vitro cleavage reactions were performed with 125 ng of exonuclease-treated circularized DNA, 90 nM of SpCas9 protein (NEB), NEB buffer 3.1 (NEB) and 270 nM of sgRNA, in a 50 μL volume. Cleaved products were A-tailed, ligated with a hairpin adaptor (NEB), treated with USER™ enzyme (NEB), and amplified by PCR with barcoded universal primers NEBNext® Multiplex Oligos for Illumina® (NEB), using Kapa HiFi Polymerase (KAPA Biosystems). Libraries were quantified by qPCR (KAPA Biosystems) and sequenced with 151 bp paired-end reads on an Illumina® NextSeq instrument. CHANGE-seq data analyses were performed using open-source CHANGE-seq analysis software.

To determine the indel frequency at CHANGE-seq-identified off-target sites, on and off-target sites for were amplified from iPSC genomic DNA obtained using rhAmpSeg™ system (IDT), and sequencing libraries were generated according to the manufacturer's instructions. Sequencing was then performed with 150-bp paired-end reads on an Illumina® Miniseq instrument. Analysis was performed using CRISPAltRations: IDT rhAmpSeq CRISPR analysis tool.

Nuclei isolation and single-cell ATAC sequencing: Isolation, washing, and counting of nuclei suspensions were performed according to the Demonstrated Protocol: Nuclei Isolation for Single Cell ATAC Sequencing (10× Genomics; CG000169 Rev D). Briefly, 1,50,000 cells were added to a 2 mL microcentrifuge tube and centrifuged at 300 g for 45 min at 4° C. The supernatant was removed without disrupting the cell pellet, and 100 μL chilled Lysis Buffer (10 mM Tris-HCl (pH 7.4), 10 mM NaCl, 3 mM MgCl₂, 0.1% Tween-20, 0.1% Nonidet® P40 Substitute, 0.01% digitonin and 1% BSA) was added and mixed 10 times. The microcentrifuge tube was then incubated on ice for 5 min. Following lysis, 1 ml chilled Wash Buffer (10 mM Tris-HCl (pH 7.4), 10 mM NaCl, 3 mM MgCl₂, 0.1% Tween-20 and 1% BSA) was added and the resulting solution was mixed 5 times. Nuclei were centrifuged at 500 g for 5 min at 4° C. and the supernatant was removed without disrupting the nuclei pellet. Nuclei were resuspended in chilled Diluted Nuclei Buffer (10× Genomics; 2000153) at approximately 6,000 nuclei per μL. The resulting nuclei concentration was then determined with a Countess® II FL Automated Cell Counter using trypan blue inclusion. Nuclei were then immediately used to generate scATAC-seq libraries according to the Chromium Single Cell ATAC Reagent Kits User Guide v1.1 (10× Genomics; 000209 Rev D). Libraries were sequenced using the Illumina NovaSeq 6000 system using S1 flow cell and target depth of 30k-37k reads/nuclei. FASTQ files were aligned with Cellranger ATAC 2.0 to custom reference Human GRCh38 genome. Downstream analyses were performed using the ArchR software package in R (Code 1).

RNA extraction and Bulk RNA sequencing: RNA was extracted from iPSCs in one well of 6-well plate using the GenElute™ Total RNA Purification Kit (Millipore Sigma) according to the manufacturer's instructions. RNA was then quantified using Nanodrop™ 2000 (ThermoFisher Scientific) and the integrity of samples were confirmed with the following three criteria for inclusion: 1) a concentration>50 ng/ml, 2) an A260/A280 rating of 1.8 and 2.1, and 3) an A260/A230 ratio>1.8. Total RNA was submitted to the University of Wisconsin-Madison Biotechnology Center and was verified for purity and integrity via the NanoDrop™ One Spectrophotometer and Agilent 2100 BioAnalyzer, respectively. Samples that met the Illumina® sample input guidelines were prepared according to the TruSeq®Stranded Total Sample Preparation Guide (1000000040499 v00) using the Illumina® TruSeq ® Stranded Total Sample Preparation kits (Illumina Inc., San Diego, Calif., USA) and sequenced on an Illumina® NovaSeg™ 6000 system at a run length 2×150 bp and target read depth of 30 million reads/sample at the University of Wisconsin—Madison Biotechnology Center. For analysis, alignments and gene counts were generated against GENCODE human transcriptome version 37 using Salmon. Transcripts were annotated to the gene level using the tximport package in and exported to CSV format (Code 2). Normalized counts were then extracted and genes with read counts <10 were filtered out. Fold change was then calculated as the ratio of the reads of the TSA-treated iPSCs to the untreated iPSCs.

Antibodies and Staining: All cells were fixed for 15 minutes with 4% paraformaldehyde in PBS (Sigma-Aldrich, St. Louis, Mo.) and permeabilized with 0.5% Triton™-X (Sigma-Aldrich) for >4 hours at room temperature before staining. Hoechst (H1399; Thermo Fisher Scientific, Waltham, Mass.) was used at 5 μg/mL with 15 min incubation at room temperature to stain nuclei. Primary antibodies were applied overnight at 4° C. in a blocking buffer of 5% donkey serum (Sigma-Aldrich) at the following concentrations: H3K9Me3 (ab8898; Abcam) 1:500; H3K9Ac (39918; Active Motif) 1:100; H3K27Me3 (C36B11; Cell Signaling Technology) 1:200. Secondary antibodies were obtained from Thermo Fisher Scientific and applied in a blocking buffer of 5% donkey serum for one hour at room temperature at concentrations of 1:400-1:800. A Nikon Eclipse Ti epifluorescence microscope was used to acquire 100× images. Images were processed using image analysis software CellProfiler to calculate the chromatin condensation values.

Karyotyping: Cells cultured for at least 5 passages were grown to 60-80% confluence and shipped for karyotype analysis to WiCell Research Institute, Madison, Wis. G-banded karyotyping was performed using standard cytogenetic protocols. Metaphase preparations were digitally captured with Applied Spectral Imaging software and hardware. For each cell line, 20 GTL-banded metaphases were counted, of which a minimum of 5 were analyzed and karyotyped. Results were reported in accordance with guidelines established by the International System for Cytogenetic Nomenclature 2016.

Statistical Analysis: Unless otherwise specified, p-values were calculated using a non-parametric Kruskal-Wallis test for multiple unmatched comparisons with GraphPad Prism software. Statistical tests were deemed significant at α≤0.05. Technical replicates are defined as distinct wells within an experiment. Biological replicates are experiments performed with different passages of iPSCs. No a priori power calculations were performed.

gRNAs and primer sequences are provided in Table 1. Top off-target sites for HIST1H2BJ-GFP, AAVS1 S10, and EMX1 are provided in Tables 2-4. Primers for PCR amplification around these off-target sites in Tables 2-4 were designed according to rhAMP protocols (Integrated DNA Technologies, Coralville, Iowa).

TABLE 1
GRNA AND PRIMER SEQUENCES.

| | | SEQ | NGS | SEQ | NGS | SEQ |
|---|---|---|---|---|---|---|
| Target | gRNA | ID | Forward Primer | ID | Reverse Primer | ID |
| Locus | (5′ to 3′) | NO: | (5′ to 3′) | NO: | (5′ to 3′) | NO: |
| HIST1H2B- | GCTGAAGCACT | 4 | CGGCAAGCTGACC | 5 | CGTCCAGGAGCG | 6 |
| GFP | GCACGCCGT | | CTGAAGTTC | | CACCATCTTC | |
| EMX1 | GAGTCCGAGCA | 7 | CAAAGTACAAACG | 8 | GTTGCCCACCCTA | 9 |
| | GAAGAAGAA | | GCAGAAGC | | GTCATTG | |
| VEGFA | GGGTGGGGGGA | 10 | AAGCAACTCCAGT | 11 | CCCTAGTGACTGC | 12 |
| | GTTTGCTCC | | CCCAAAT | | CGTCTG | |
| AAVS1 S8 | GCTGTCCTGAA | 13 | TTGCCTGGACACC | 14 | GCCACATTAACC | 15 |
| | GTGGACATA | | CCGTTCTCCT | | GGCCCTGGGAA | |
| AAVS1 S10 | GGGAACCCAGC | 16 | CTTCCTTCTCGGCG | 17 | AGCCAGGGAGAC | 18 |
| | GAGTGAAGA | | CTGCACCAC | | GGGGTACTTTGG | |
| TRAC S2 | GCTGGTACACG | 19 | GCCTGGGTTGGGG | 20 | GTTGCTCCAGGCC | 21 |
| | GCAGGGTCA | | CAAAGAGGGA | | ACAGCACTGT | |
| TRAC S3 | GAGAATCAAAA | 22 | GCCTGGGTTGGGG | 23 | GTTGCTCCAGGCC | 24 |
| | TCGGTGAAT | | CAAAGAGGGA | | ACAGCACTGT | |

TABLE 2
TOP 12 OFF-TARGET SITES FOR HIST1H2B-GFP LOCUS OBTAINED BY
CHANGE-SEQ IN iPSCs.

| | | | | | CHANGE- | | | |
|---|---|---|---|---|---|---|---|---|
| | | | | | SEQ | | SEQ | |
| | #Chromo- | | | | Read | | ID | |
| Name | some | Start | End | Strand | Count | Site_Sequence | NO: | Cells |
| OT1 | chr19 | 43463243 | 43463266 | + | 2452 | GCTGTAGCACTCCACGCCGTTGG | 25 | hiPSC |
| OT2 | chr9 | 98626054 | 98626076 | + | 1030 | | | hiPSC |
| OT3 | chr3 | 73404993 | 73405016 | - | 548 | AGCAAAGCACTGCACACAGTGGG | 26 | hiPSC |
| OT4 | chr5 | 6422338 | 6422361 | - | 524 | CAGGAAGCACTGCACACTGTGGG | 27 | hiPSC |
| OT5 | chr8 | 51474994 | 51475018 | + | 338 | | | hiPSC |
| OT6 | chr4 | 5703158 | 5703181 | - | 322 | AAAGAAGCACTGCATGCTGTAGG | 28 | hiPSC |
| OT7 | chr1 | 89051013 | 89051036 | + | 300 | CCTGCACCCTTGCACGCCATTGG | 29 | hiPSC |
| OT8 | chr5 | 151028994 | 151029017 | - | 274 | GAAGAAGCACCACACACAGTAGG | 30 | hiPSC |
| OT9 | chr13 | 114129061 | 114129084 | + | 254 | GAGGAGGCACTGCACGCCTTGGG | 31 | hiPSC |
| OT10 | chr16 | 489418 | 489441 | + | 254 | ATAGAAGCACAACACGCAGTGGG | 32 | hiPSC |
| OT11 | chr9 | 37922745 | 37922768 | + | 252 | GCTCAAGCACTGCACCCCGTGGG | 33 | hiPSC |
| OT12 | chr13 | 25135132 | 25135155 | - | 200 | AGTGAAGCAAAACACACCGTAGG | 34 | hiPSC |

TABLE 3
TOP 10 OFF-TARGET SITES FOR AAVSI S10 LOCUS OBTAINED BY
CHANGE-SEQ IN T-CELLS

| | | | | | CHANGE- | | | |
|---|---|---|---|---|---|---|---|---|
| | | | | | SEQ | | SEQ | |
| | #Chromo- | | | | Read | | ID | |
| Name | some | Start | End | Strand | Count | Site_Sequence | NO: | Cells |
| OT1 | chr1 | 204249266 | 204249289 | + | 590 | AGGGACCCAGAGAGTGGAGAAGG | 35 | T-cells |
| OT2 | chr11 | 34105752 | 34105775 | - | 408 | GGGATCCCAGCCAGTGGAGAAGG | 36 | T-cells |
| OT3 | chr17 | 7446661 | 7446684 | + | 376 | TGGATCCCAGCGAGTGAAGGCGG | 37 | T-cells |
| OT4 | chr12 | 49031404 | 49031427 | - | 280 | GGGAGCCCAGTCAGTGAAGAGGG | 38 | T-cells |
| OT5 | chr9 | 99045374 | 99045397 | - | 184 | AGGAGCCCAGAGAGAGAAGAGGG | 39 | T-cells |
| OT6 | chr9 | 107554249 | 107554272 | + | 178 | AAGAACACAGAGAGTGAAGAGAG | 40 | T-cells |
| OT7 | chr10 | 132440931 | 132440954 | - | 142 | GGGAACCCATGGAGTGGAGATGG | 41 | T-cells |
| OT8 | chr2 | 96757784 | 96757807 | + | 126 | GGAGACCCAGAGGGTGAAGAGGG | 42 | T-cells |
| OT9 | chr19 | 50199428 | 50199451 | - | 118 | GGGAACCCAGACAGTGAAGGGGG | 43 | T-cells |
| OT10 | chr9 | 70982063 | 70982085 | - | 106 | GAGAACCCAG-GAGTGAAGTTGG | 44 | T-cells |

TABLE 4
TOP 7 OFF-TARGET SITES FOR EMX/LOCUS OBTAINED BY GUIDE-SEQ
IN U2OS CELLS.

| | | | | | GUIDE- | | | |
|---|---|---|---|---|---|---|---|---|
| | | | | | SEQ | | SEQ | |
| | #Chromo- | | | | Read | | ID | |
| Name | some | Start | End | Strand | Count | Site_Sequence | NO: | Cells |
| OT1 | chr5 | 45359060 | 45359083 | - | 3123 | GAGTTAGAGCAGAAGAAGAAAGG | 45 | U2OS |
| OT2 | chr15 | 44109746 | 44109769 | + | 1445 | GAGTCTAAGCAGAAGAAGAAGAG | 46 | U2OS |
| OT3 | chr2 | 219845055 | 219845078 | + | 700 | GAGGCCGAGCAGAAGAAAGACGG | 47 | U2OS |
| OT4 | chr8 | 128801241 | 128801264 | + | 390 | GAGTCCTAGCAGGAGAAGAAGAG | 48 | U2OS |
| OT5 | chr5 | 9227145 | 9227168 | + | 258 | AAGTCTGAGCACAAGAAGAATGG | 49 | U2OS |
| OT6 | chr5 | 146833183 | 146833206 | - | 143 | GAGCCGGAGCAGAAGAAGGAGGG | 50 | U2OS |
| OT7 | chr1 | 23720611 | 23720634 | - | 102 | AAGTCCGAGGAGAGGAAGAAAGG | 51 | U2OS |

### EXAMPLE 1

### Optimization of Trichostatin A Treatment for IPSC Gene Editing

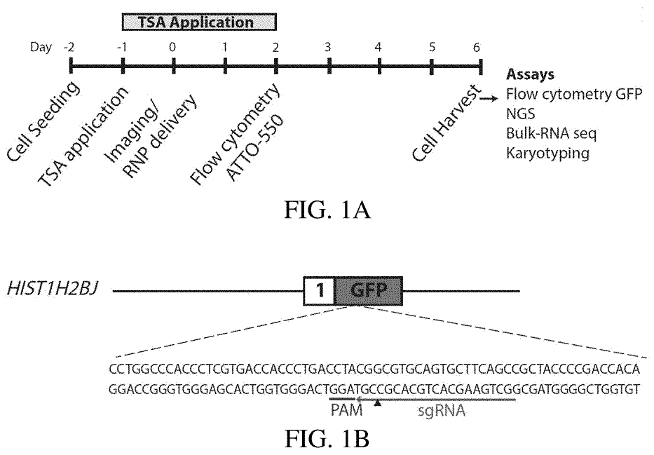

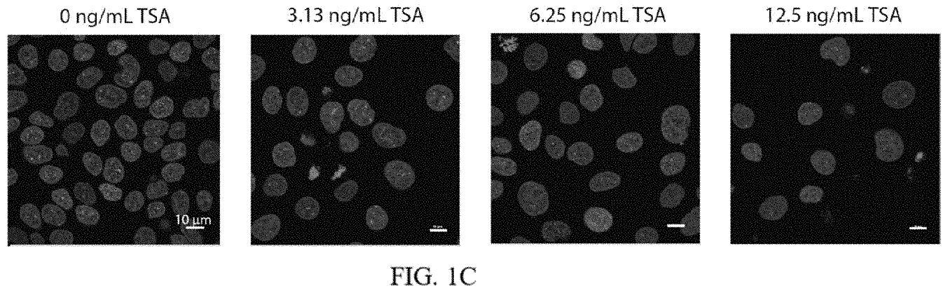

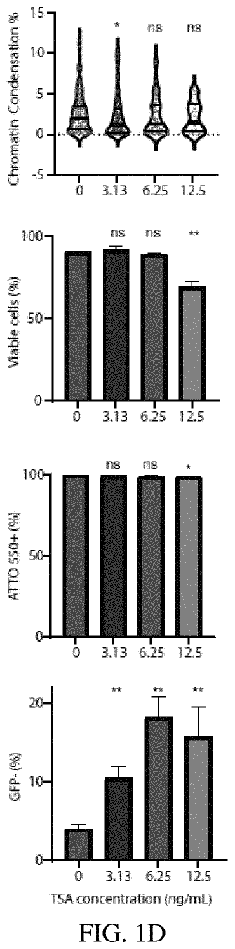

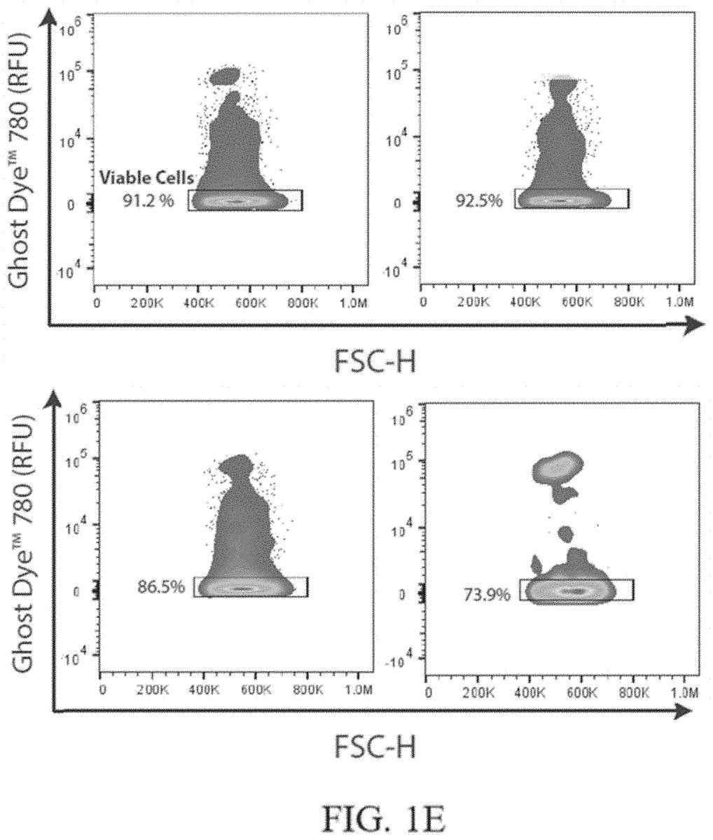

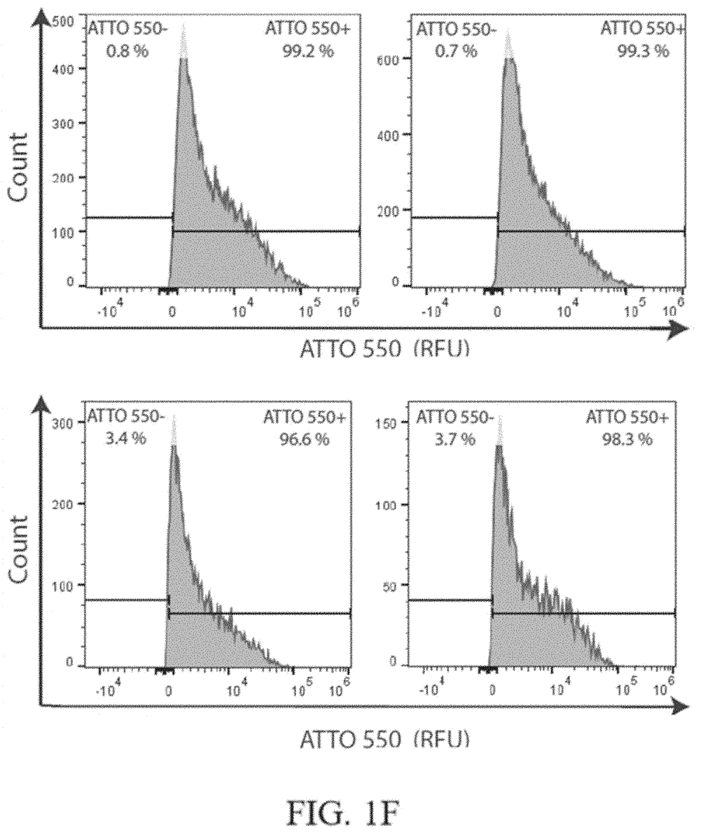

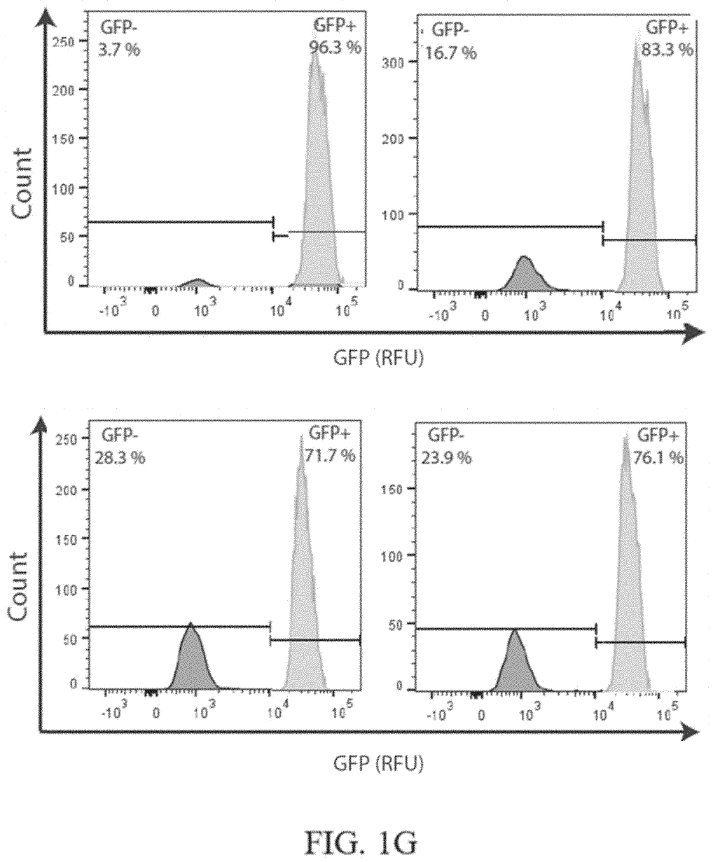

A simple, yet robust, workflow has been developed to introduce mutations in iPSCs without extensive use of deep sequencing (FIG. 1A). A mono-allelic mEGFP tagged HIST1H2BJWTC-11 human iPSC line (tag at C-terminus) was used as it provides two-fold advantages. First, targeting the mEGFP locus (referred as HIST1H2BJ-GFP) allows for easy assessment of iPSC editing efficiency via fluorescence imaging or flow cytometry, since the percentage of GFP—cells showed a strong linear correlation with the indel percentage determined by deep sequencing of genomic DNA (data not shown; R2=0.9307). Second, these iPSCs enable in situ live imaging of their cell nuclei to monitor chromatin changes during the process of CRISPR-Cas9 gene editing. An Alt-R® CRISPR-Cas9 2-part single guide RNA (sgRNA) system (IDT) was employed, which consists of 1) crRNA sequence to target the mEGFP locus in iPSCs (FIG. 1B), and 2) ATTO™ 550 fluorescent dye labelled tracrRNA that binds to Cas9. A range of TSA concentrations (0-200 ng/mL) were tested on iPSCs to determine the effect of TSA on cell viability. Cell viability decreased with the increase in TSA concentration (data not shown), with a considerable decrease for TSA concentration>25 ng/mL. TSA concentrations of 0, 3.13, 6.25, and 12.5 ng/mL for further studies. Next, a range of TSA treatment durations (0-24 hours) were assessed and two methods for delivery for the Cas9 RNP complex (lipofection and electroporation) were also assessed to determine the optimal conditions for gene editing. Since TSA treatment duration of 20 hours (data not shown) and lipofection (data not shown) yielded the highest gene editing efficiencies, these conditions were implements for subsequent experiments.

### EXAMPLE 2

### Trichostatin A Enhances Gene Editing Efficiency of IPSCS

After streamlining the gene editing strategy, the effect of TSA treatment on the gene editing efficiency of iPSCs was examined The iPSCs were pre-treated with TSA (0, 3.13, 6.25, and 12.5 ng/mL) and their nuclei imaged. Sequencing data analyzed using the CRISPAltRations tool, which allowed for quantitative analysis of observed indel mutations and their spatial distribution in the target region The images were then used as inputs for a CellProfiler software pipeline to output the chromatin condensation % of the nuclei (data not shown), defined as the ratio of total heterochromatin intensity to the total nuclear intensity. Immunofluorescence labeling validated the heterochromatin foci identified in the GFP images by the CellProfiler pipeline i.e., the H3K9Ac euchromatin histone mark was excluded from the heterochromatin foci, while the H3K9Me3 heterochromatin histone mark overlapped with the heterochromatin foci (Data not shown). Thus, chromatin condensation % is representative of the global chromatin state of cells.

The nuclear image analysis pipeline revealed a decrease in chromatin condensation% upon TSA treatment (FIG. 1C,D), indicating that the nuclear imaging pipeline is sensitive to the chromatin decondensation induced by TSA. The dose dependence of Cas9 RNP transfection efficiency (% ATTO™ 550+cells/viable cells) was assessed by performing flow cytometry analysis on day 2 after Cas9 RNP delivery. High transfection efficiencies >95% (FIG. 1F) were noted, indicating successful delivery of Cas9 RNP independent of the TSA concentration. Furthermore, flow cytometry analysis on day 6 after Cas9 RNP delivery revealed a positive correlation between that the gene editing efficiency (% GFP—cells/viable cells) and the dose of TSA for concentrations up to 6.25 ng/mL (FIG. 1G). While increasing the concentration of TSA to 6.25 ng/mL showed approximately a 2-3 fold increase in gene editing efficiency, an additional increase in the TSA concentration to 12.5 ng/mL did not increase the editing efficiency further. Without being held to theory, it was hypothesized that this could be due to the higher cell toxicity that occurred at 12.5 ng/mL TSA concentration (FIG. 1E). Taken together, these results indicate that nuclear imaging is sensitive to the chromatin condensation changes induced by TSA and that TSA enhances CRISPR-Cas9 mediated gene editing in a dose-dependent manner

### EXAMPLE 3

### Deep Sequencing of Edited IPSCS Reveals Increased Gene Editing and Changes in Indel Profiles at Multiple Loci Upron TSA Application

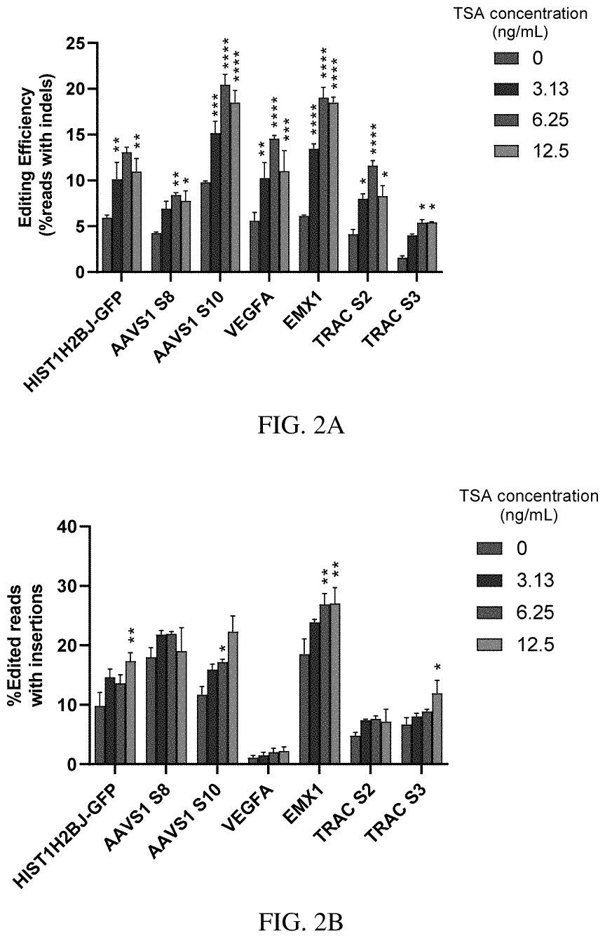

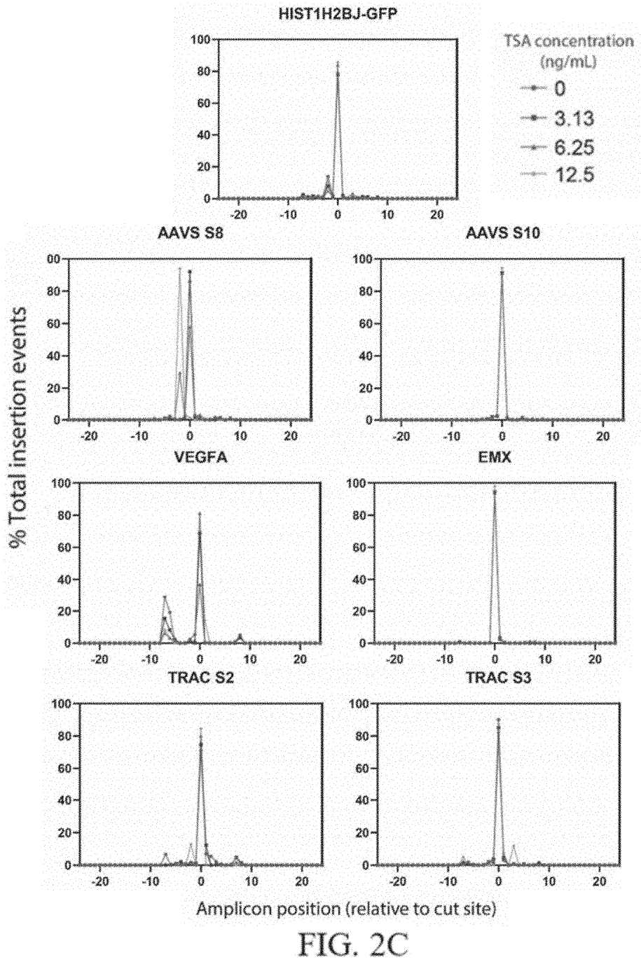

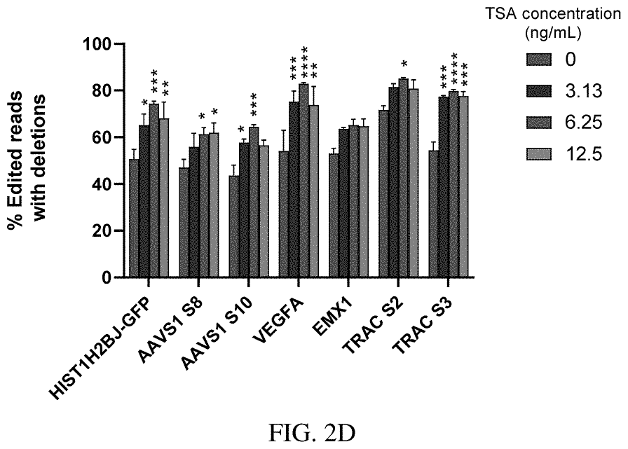

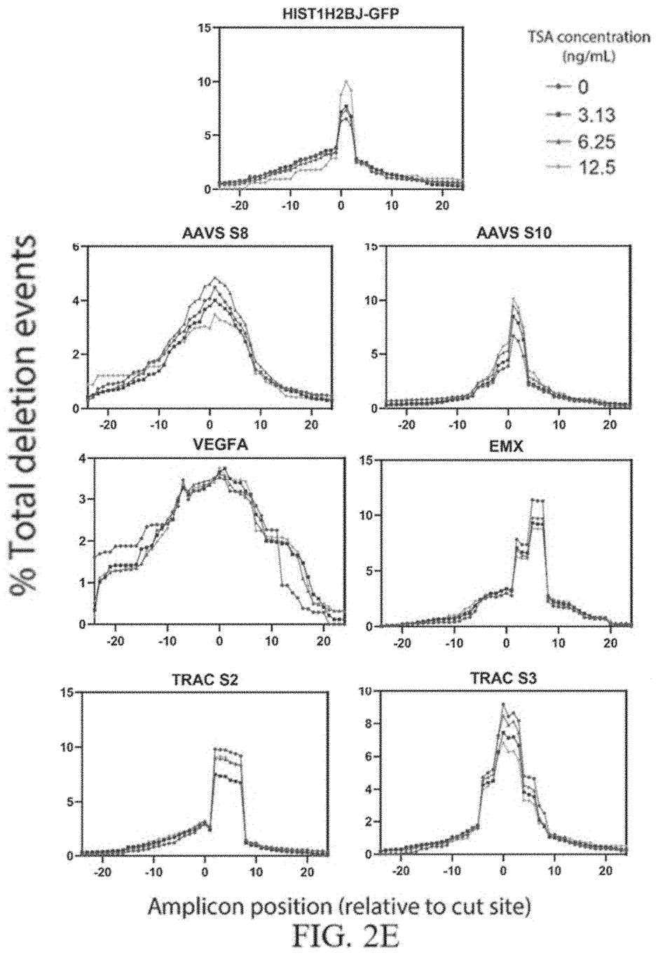

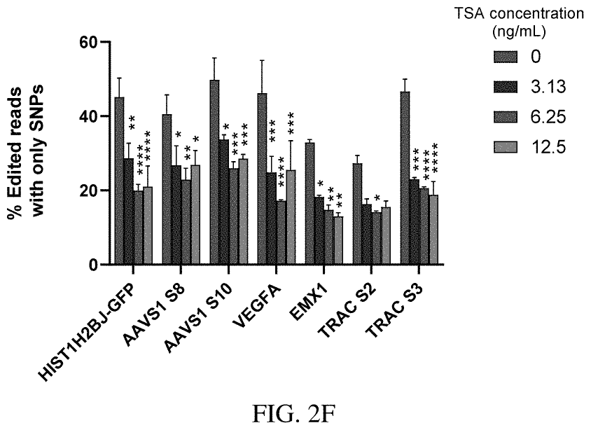

Multiple gRNA sequences targeting different sites of open (HIST1H2BJ and AAVS1) and closed chromatin (TRAC, VEGFA, EMX1) were selected to investigate if the observed TSA-induced increase in gene-editing efficiency at HIST1H2BJ-GFP locus is specific to the gRNA sequence or the chromatin state of the target locus. To gain a more detailed analysis of gene-edited events in iPSCs, deep sequencing was performed on edited cells obtained after TSA treatment. The genomic DNA was harvested on day 6 after Cas9 RNP delivery. PCR using primers flanking the gRNA target sites was then performed and prepared for next-generation sequencing (NGS) using the Illumina® MiniSeq™ system. TSA treatment increased gene-editing efficiency at all the sites independent of the gRNA sequence and initial chromatin state of the loci (FIG. 2A). Corroborating with this observation, we noted an increase in the local chromatin accessibility at the on-target sites upon TSA treatment (Data not shown). Moreover, the percentage of edited reads containing insertion events did not undergo significant change and deletion events decreased upon TSA treatment (FIG. 2B,D), while the insertion and deletion profiles around the cut site were similar (FIG. 2C,E). The highest frequency of indels was found three to four nucleotides upstream from the protospacer adjacent motif (PAM) sequence, consistent with reports of type II CRISPR systems (FIG. 2C,E). Furthermore, edited reads contained higher deletion events (approximately 50-80%) than insertion events (approximately 5-20%). Similar observations were reported in deep sequencing analysis of human cells edited by S. Pyogenes Cas9 (SpyCas9). The percentage of edited reads with Single Nucleotide Polymorphisms (SNPs) decreased upon chromatin decondensation via TSA treatment (FIG. 2F), while the relative distribution of SNPs around the cut site remained similar (data not shown). This is consistent with reports that indicate nucleosomes are enriched in SNPs. Taken together, these results indicate that TSA treatment leads to shifts in indel profiles.

### EXAMPLE 4

### Off-Target Profiling Edited IPSCS

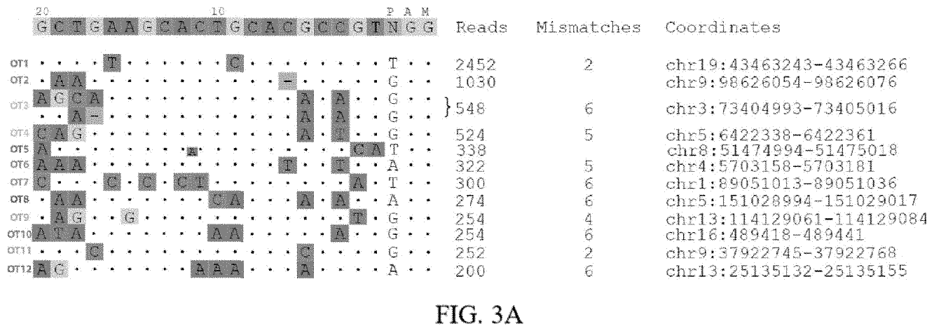

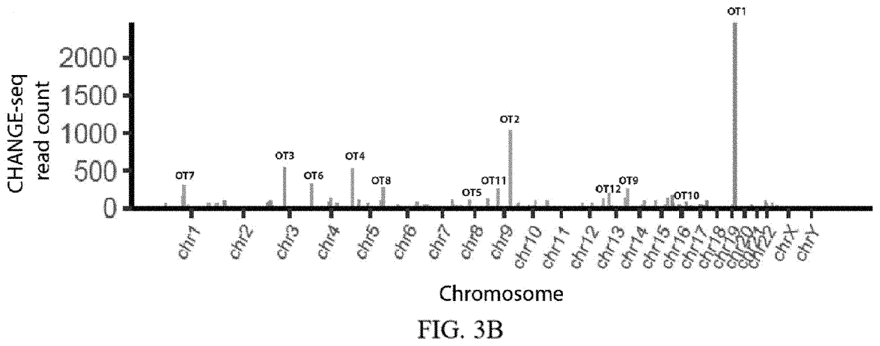

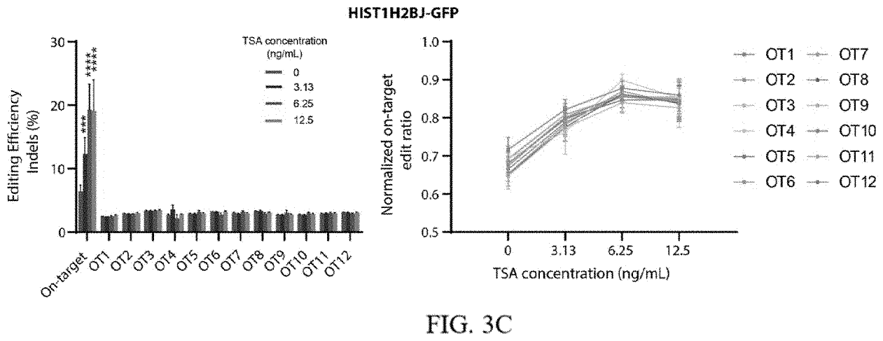

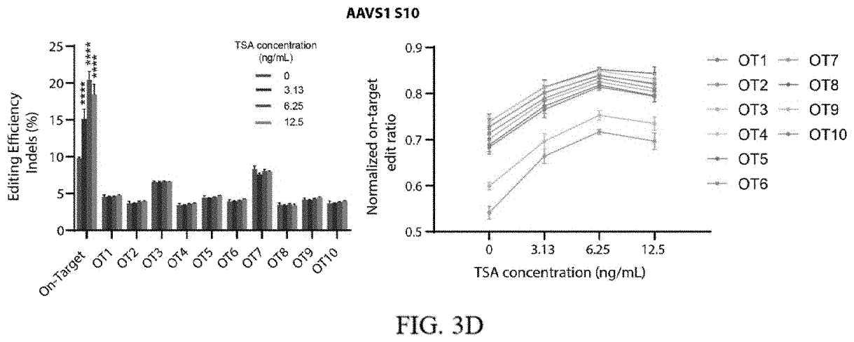

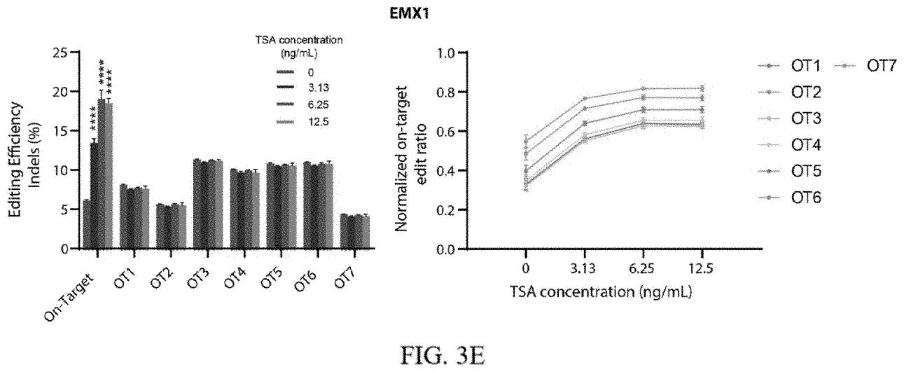

Highly sensitive genome-wide, off-target analysis for our TSA-based editing strategy was assayed by CHANGE-seq at HIST1H2BJ-GFP site, which yielded a frequency distribution of the potential off-target sites (FIG. 3A,B). Top 12 off-target sites were amplified from genomic DNA using the rhAmp-Seq system (IDT) and subsequently sequenced using the Illumina® MiniSeq™ system. Sequencing data was then analyzed using the CRISPAltRations tool, which allowed for quantitative analysis of observed indel mutations (data not shown). Indel reads at off-target sites primarily constituted of SNPs and indel % at off-target sites did not increase upon TSA treatment (FIG. 3C). Furthermore, the highest normalized on-target edit ratio was obtained for TSA concentration of 6.25 ng/mL, representative of the optimal TSA concentration for generating gene editing iPSCs. We additionally performed similar off-target analysis at top off-target sites for AAVS1 S10 and EMX1 loci, which showed no significant increase in indel % at off-target sites and highest normalized on-target edit ratio at TSA concentration of 6.25 ng/mL (FIG. 3D,E) similar to HIST1H2B-GFP site. Corroborating with this observation, no significant increase in local chromatin accessibility at the off-target sites upon TSA treatment was noted (data not shown).

To further study the impact of TSA on iPSCs, we performed bulk-RNA sequencing on TSA-treated iPSCs, and preliminary analysis showed that there were several upregulated and downregulated genes upon TSA treatment (data not shown), which are involved in cell cycle regulation (MAGEA4, DHRS2), cell signaling (STMN2) and cell growth regulation (ERBB2, AHRR). A more detailed investigation with more replicates can allude to pathways involved in TSA-induced increase in gene editing efficiency.

### EXAMPLE 5

### Karyotypic Analysis of Edited IPSCS

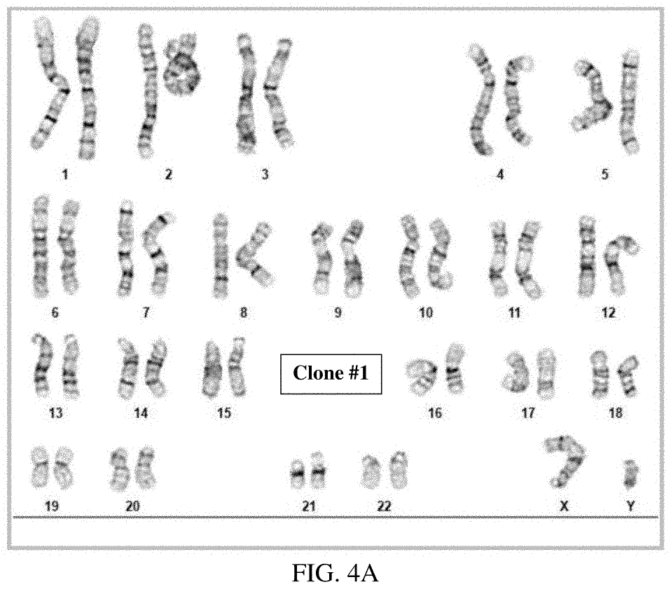

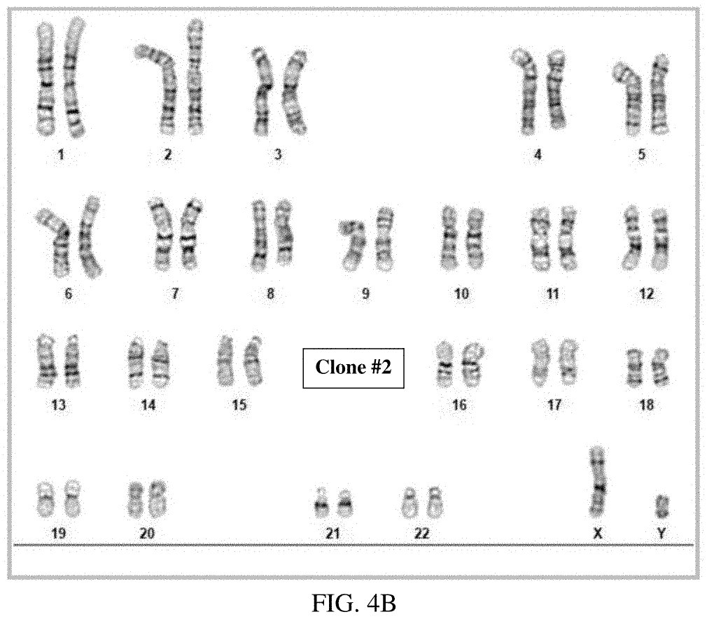

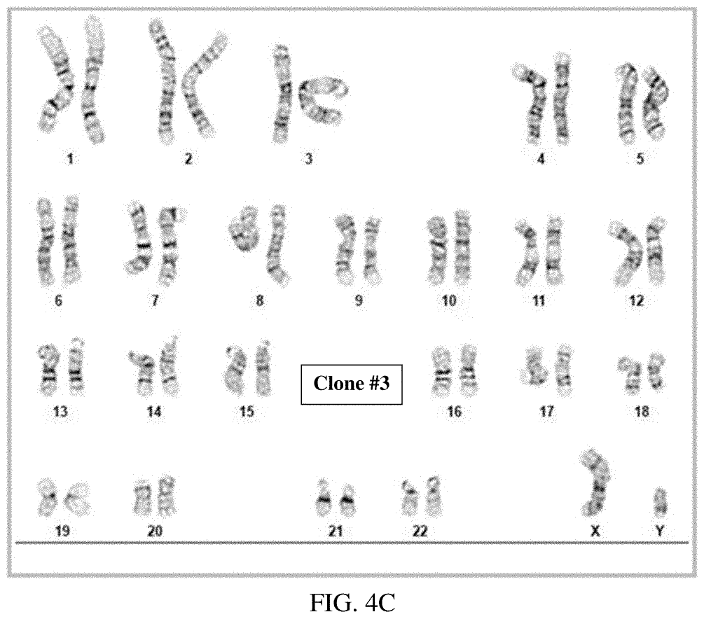

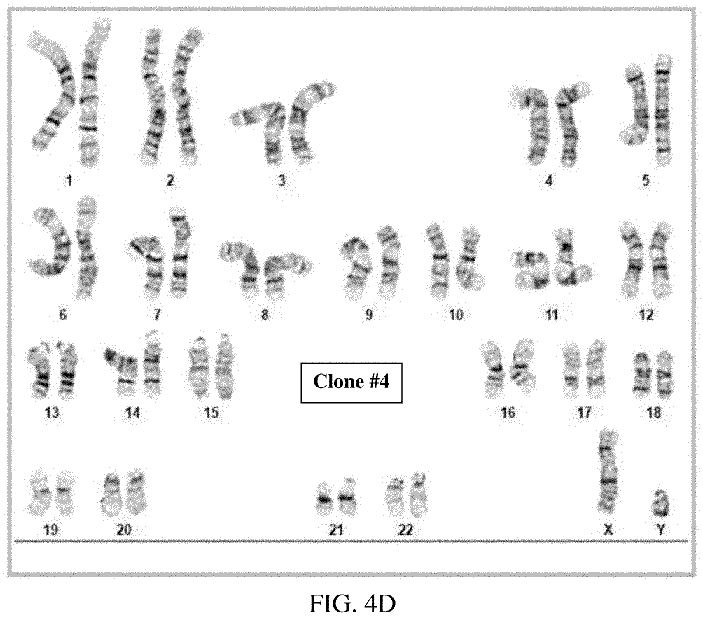

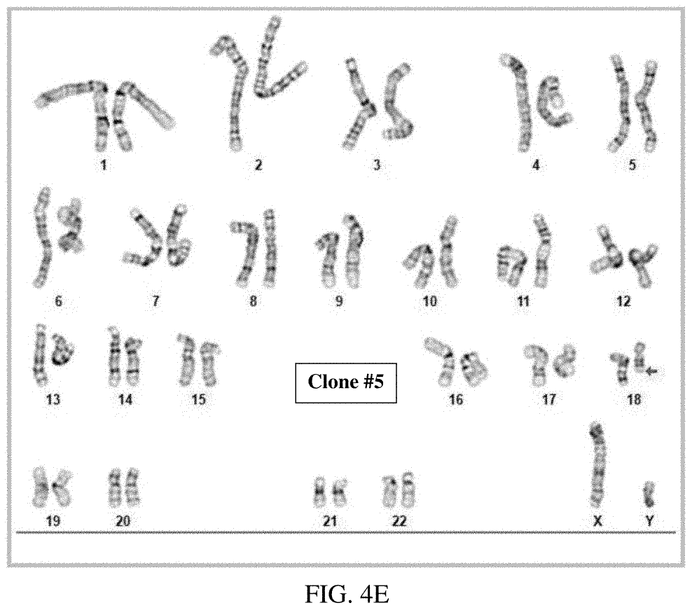

Multiple gene-edited iPSC clones were isolated from the 6.25 ng/mL TSA treatment condition at HIST1H2BJ-GFP locus and expanded for subsequent characterization for pluripotency and any genomic abnormalities. Bulk-RNA sequencing (data not shown) indicated that TSA treatment did not decrease gene expression of pluripotency marker genes (OCT4, NANOG, SOX2, TRA-1-60), indicating that TSA treatment does not affect the pluripotency of iPSCs. Besides, no changes were observed in iPSC morphology during long-term culture of the edited cells, indicating that TSA treatment does not cause aberrations in iPSC morphology. Karyotyping analysis indicated no genomic abnormalities in four of the five clones (FIG. 4A-E), while one clone showed an interstitial duplication in the long (q) arm of chromosome 20 in five of the twenty cells examined (FIG. 4E). This is a known recurrent acquired duplication at this location in human pluripotent stem cell cultures, and did not consistently arise in all of our edited lines.

Discussion: Overall, we described an easy and rapid chromatin modulation-based CRISPR-Cas9 workflow to manufacture gene-edited iPSCs efficiently. TSA (HDAC inhibitor) was used as the chromatin modulator, TSA-induced chromatin decondensation was quantified using nuclear imaging, and multiple open and closed chromatin loci in HIST1H2BJ tagged mEGFP reporter iPSCs were subsequently edited. Relative to the traditional CRISPR-Cas9 gene editing workflow, the streamlined manufacturing workflow described herein can: 1) increase the gene-editing efficiency up to approximately 2-3 fold while ensuring no increased off-target mutations, and maintenance of pluripotency and genomic integrity; and 2) identify iPSCs more amenable to gene editing based on nuclear imaging outputs, thus enabling the reduction in cost and times associated with culturing and passaging to isolate rare edited iPSC clones.

To gain deeper insights into the mechanisms involved in TSA-induced gene editing and push capabilities of this strategy, performed scATAC and bulk-RNA sequencing experiments were performed to identify genome-wide changes in chromatin accessibility and gene expression. While our imaging pipeline revealed TSA-induced chromatin condensation changes at the global level, scATAC-seq analysis revealed local changes in chromatin accessibility at different regions in the genome (data not shown). Moreover, preliminary bulk-RNA sequencing revealed several differentially regulated genes (data not shown) across the genome. The kinetics of SpCas9 RNP binding at off-target sites are known to be different from those at the on-target site, and thus strong target strand recognition at the on-target site may be involved in the mechanisms by which TSA promotes editing. Further, the kinetics of DNA repair processes may be different form the reduced level of DNA double strand break or nick formation at the off-target site. These processes may also be affected by TSA. However, given the large conservation of mutational spectrum at most of the sites evaluated over all TSA treatments, it is unlikely that TSA is globally switching end joining DNA repair pathways.

Additionally, the workflow described herein enables the assessment of other biological processes like cell proliferation, apoptosis, and DNA damage via imaging of GFP, CellEvent Caspase 3/7 Detection reagent, and immunostained histone H2AX phosphorylation, respectively. Thus, the action of small molecules or any other perturbation and the impacted processes could be tracked in parallel in each well via high-content nuclear imaging This study can also be extended in the future to study NHEJ vs HDR (using an HDR activated GFP to BFP switch), to other natural or engineered Cas enzyme variants, to other epigenetic modifiers using GFP tagging approach, and to other cell types including primary cells, cardiomyocytes, T-cells, and neural progenitors. Finally, since iPSCs are a heterogeneous population, studying changes at the single-cell level may help elucidate genome editing mechanisms more than looking at bulk level data. scATAC-seq data generated from transfected iPSCs can be coupled to genome editing outcome (via genotyping of transcriptomes) for a single cell level look at the differences in the transcriptional program between edited and unedited cells. The use of single-cell sequencing-based techniques may also be useful where GFP tagging or confocal imaging is not facile (e.g., primary T-cells).

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. “About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. An ex vivo method of site-specifically editing a target cell genome, comprising treating a population of unmodified target cells with a Class I and/or Class II histone deacetylase inhibitor to provide a population of chromatin decondensed unmodified target cells; and
introducing into the population of chromatin decondensed unmodified target cells a Cas9 ribonucleoprotein, to provide a population of site-specifically genome-edited target cells;
wherein the Cas9 ribonucleoprotein comprises a Cas9 protein and a guide RNA and cleaves DNA at a cleavage site in the target cell genome.

2. The method of claim 1, wherein the cleavage site in the target cell genome is in a target cell expressed gene, a regulatory region, noncoding RNA, repeat region, integrated viral genome, topologically associating domain, or a lamina-associated domain.

3. The method of claim 1, wherein the cleavage site in the target cell genome creates a precise knockout in the target cell genome.

4. The method of claim 3, wherein the precise knockout of the donor cell expressed gene proceeds primarily by a non-homologous end-joining repair pathway (NHEJ).

5. The method of claim 1, further comprising introducing into the population of chromatin decondensed unmodified target cells a donor polynucleotide comprising a synthetic DNA sequence flanked by homology arms that are complementary to sequences on both sides of the cleavage site in the target cell genome, wherein the synthetic DNA sequence in the donor polynucleotide is specifically integrated into the cleavage site of the target cell genome.

6. The method of claim 5, wherein the donor polynucleotide includes a mutation, deletion, alteration, integration, gene correction, gene replacement, transgene insertion, nucleotide deletion, gene disruption, a gene mutation, or a combination thereof.

7. The method of claim 1, wherein the guide RNA is a one-part sgRNA or a two-part guide RNA comprising a crRNA and a tracrRNA.

8. The method of claim 1, further comprising live in situ nuclear imaging of the population of chromatin decondensed unmodified target cells, and quantifying the histone deacetylase inhibitor-induced decrease in chromatin condensation of the target cell nuclei.

9. The method of claim 8, wherein the histone deacetylase inhibitor-induced decrease in chromatin condensation percentage is at least a 1% decrease in chromatin condensation percentage calculated as a percentage of heterochromatin intensity to the total nuclear intensity.

10. The method of claim 1, wherein the target cells comprise induced pluripotent stem cells (iPSCs), hematopoietic stem cells (HPSCs), neural progenitor cells, embryonic stem cells, natural killer cells (NK cells), or T cells.

11. The method of claim 1, wherein a percentage of off-target genome edited sites is less than half of a percentage of on-target genome edits.

12. The method of claim 1, wherein the Class I and/or Class II histone deacetylase inhibitor is vorinostat, panobinostat, belinostat, entinostat, phenyl butyrate, valproic acid, trichostatin A, mocetinostat, pracinostat, dacinostat, givinostat, abexinostat, depsipeptide, or a combination thereof.

13. The method of claim 1, wherein the Class I and/or Class II histone deacetylase inhibitor is trichostatin A, and treating a population of unmodified target cells with the Class I and/or Class II histone deacetylase inhibitor is done for 16 to 24 hours at a temperature of 37° C., and a concentration of 3.125 ng/mL to 12.5 ng/mL.

14. The method of claim 1, further comprising selecting a clone from the population of genome-edited target cells and expanding the clone to provide a population of clonally expanded genome-edited cells.

15. The method of claim 14, wherein the gene expression of pluripotency genes OCCT4, NANOG, SOX2, and TRA-1-60 is decreased by less than 50% in the clonally expanded genome-edited cells compared to clonally expanded genome-edited cells produced in the absence of the II histone deacetylase inhibitor.

16. The method of claim 14, further comprising differentiating the population of clonally expanded genome-edited cells to provide a population of differentiated genome-edited cells.

17. A method of allogenic or autologous cell therapy comprising transplanting the differentiated genome-edited target cells of claim 16 into a subject in need thereof.

18. The method of claim 17, wherein the differentiated genome-edited cells are in the form of a transfusion, a tissue transplant, or a medical device.

19. The method of claim 18, wherein the allogenic or autologous cell therapy comprises a tissue graft, a blood transfusion, a cancer immunotherapy, a bone marrow transplant, or a combination thereof.

20. The method of claim 17, wherein the subject is treated for an inherited cardiac disease, Huntington's disease, Alzheimer's disease, Parkinson's disease, schizophrenia, amyotrophic lateral sclerosis, spinal muscular atrophy, Rett syndrome, Prader-Willi syndrome, non-Hodgkin lymphoma, Hodgkin lymphoma, leukemia, multiple myeloma, aplastic anemia, diabetes, sickle cell disease, thalassemia, lysosomal storage diseases, Duchenne's Muscular Dystrophy, inherited retinal disorder, or cystic fibrosis, cancer, kidney disease, or a liver disease.

21. A medical device comprising the differentiated genome-edited cells of claim 16.

22. An in vitro disease model comprising the genome-edited target cells of claim 1.

23. The in vitro disease model of claim 22, wherein the disease is an inherited cardiac disease, Huntington's disease, Alzheimer's disease, Parkinson's disease, schizophrenia, amyotrophic lateral sclerosis, spinal muscular atrophy, Rett syndrome, Prader-Willi syndrome, non-Hodgkin lymphoma, Hodgkin lymphoma, leukemia, multiple myeloma, aplastic anemia, diabetes, sickle cell disease, thalassemia, lysosomal storage diseases, Duchenne's Muscular Dystrophy, inherited retinal disorder, or cystic fibrosis, cancer, kidney disease, or a liver disease.

24. The in vitro disease model of claim 22, wherein the genome-edited target cells are expanded and differentiated and are alveolar epithelial cells, airway epithelial cells, neuronal cells, adipocytes, cardiomyocytes, hematopoietic cells, pancreatic beta cells, retinal epithelial cells, photoreceptors, retinal ganglion cells, epidermal cells, intestinal epithelial cells, smooth muscle cells, skeletal muscle cells, renal cells, chondrocytes, osteocytes, stromal cells, T cells, natural killer cells, macrophages, or red blood cells.

25. The in vitro disease model of claim 22, wherein the model is used for modeling disease outcome, screening a target drug, biologic or genetic medicine treatment, or testing toxic side-effects of a treatment.

26. A method of guide-RNA design, comprising
treating a population of unmodified target cells with a Class I and/or Class II histone deacetylase inhibitor to provide a population of chromatin decondensed unmodified target cells;
introducing into the population of chromatin decondensed unmodified target cells a Cas9 ribonucleoprotein, to provide a population of genome-edited target cells, wherein the Cas9 ribonucleoprotein comprises a Cas9 protein and a first guide RNA, and cleaves DNA at a cleavage site in the target cell genome;
determining the percentage of off-target genome edited sites in the population of genome-edited target cells compared to on-target genome-edited sites; and
designing a second guide RNA when the percentage of off-target genome edited sites produced by the first guide RNA is greater than half of the percentage of the on-target genome edits produced by the first guide RNA, wherein the second guide RNA is predicted to reduce off-target genome-edited sited compared to the first guide RNA.

27. The method of claim 26, further comprising designing one or more subsequent guide RNAs to the second guide RNAs, wherein the designing optimizes the percentage of off-target genome edited sites in the population of genome-edited target cells compared to on-target genome-edited sites.


SEQUENCE LISTING (55 sequences)

SEQ ID NO: 1 - DNA, 66 bp, Homo sapiens
  source 1..66: mol_type = other DNA
cctggcccaccctcgtgaccaccctgacctacggcgtgcagtgcttcagccgctaccccgaccaca

SEQ ID NO: 2 - DNA, 61 bp, Homo sapiens
  source 1..61: mol_type = other DNA
tgtggtcggggtagcggctgaagcacgccgtaggtcagggtggtcacgagggtgggccagg

SEQ ID NO: 3 - DNA, 23 bp, synthetic construct
  source 1..23: mol_type = other DNA; note = HIST1H2BJ-GFP gRNA change sequence
gctgaagcactgcacgccgtngg

SEQ ID NO: 4 - DNA, 20 bp, synthetic construct
  source 1..20: mol_type = other DNA; note = HIST1H2B-GFP gRNA
gctgaagcactgcacgccgt

SEQ ID NO: 5 - DNA, 22 bp, synthetic construct
  source 1..22: mol_type = other DNA; note = HIST1H2B-GFP NGS Forward Primer
cggcaagctgaccctgaagttc

SEQ ID NO: 6 - DNA, 22 bp, synthetic construct
  source 1..22: mol_type = other DNA; note = HIST1H2B-GFP NGS Reverse Primer
cgtccaggagcgcaccatcttc

SEQ ID NO: 7 - DNA, 20 bp, synthetic construct
  source 1..20: mol_type = other DNA; note = EMX1 gRNA
gagtccgagcagaagaagaa

SEQ ID NO: 8 - DNA, 21 bp, synthetic construct
  source 1..21: mol_type = other DNA; note = EMX1 NGS Forward Primer
caaagtacaaacggcagaagc

SEQ ID NO: 9 - DNA, 20 bp, synthetic construct
  source 1..20: mol_type = other DNA; note = EMX1 NGS Reverse Primer
gttgcccaccctagtcattg

SEQ ID NO: 10 - DNA, 20 bp, synthetic construct
  source 1..20: mol_type = other DNA; note = VEGFA gRNA
gggtggggggagtttgctcc

SEQ ID NO: 11 - DNA, 20 bp, synthetic construct
  source 1..20: mol_type = other DNA; note = VEGFA NGS Forward Primer
aagcaactccagtcccaaat

SEQ ID NO: 12 - DNA, 19 bp, synthetic construct
  source 1..19: mol_type = other DNA; note = VEGFA NGS Reverse Primer
ccctagtgactgccgtctg

SEQ ID NO: 13 - DNA, 20 bp, synthetic construct
  source 1..20: mol_type = other DNA; note = AAVS1_Site_8 gRNA
gctgtcctgaagtggacata

SEQ ID NO: 14 - DNA, 23 bp, synthetic construct
  source 1..23: mol_type = other DNA; note = AAVS1_Site_8 NGS Forward Primer
ttgcctggacaccccgttctcct

SEQ ID NO: 15 - DNA, 23 bp, synthetic construct
  source 1..23: mol_type = other DNA; note = AAVS1_Site_8 NGS Reverse Primer
gccacattaaccggccctgggaa

SEQ ID NO: 16 - DNA, 20 bp, synthetic construct
  source 1..20: mol_type = other DNA; note = AAVS1_Site_10 gRNA
gggaacccagcgagtgaaga

SEQ ID NO: 17 - DNA, 23 bp, synthetic construct
  source 1..23: mol_type = other DNA; note = AAVS1_Site_10 NGS Forward Primer
cttccttctcggcgctgcaccac

SEQ ID NO: 18 - DNA, 24 bp, synthetic construct
  source 1..24: mol_type = other DNA; note = AAVS1_Site_10 NGS Reverse Primer
agccagggagacggggtactttgg

SEQ ID NO: 19 - DNA, 20 bp, synthetic construct
  source 1..20: mol_type = other DNA; note = TRAC_Site_2 gRNA
gctggtacacggcagggtca

SEQ ID NO: 20 - DNA, 23 bp, synthetic construct
  source 1..23: mol_type = other DNA; note = TRAC_Site_2 NGS Forward Primer
gcctgggttggggcaaagaggga

SEQ ID NO: 21 - DNA, 23 bp, synthetic construct
  source 1..23: mol_type = other DNA; note = TRAC_Site_2 NGS Reverse Primer
gttgctccaggccacagcactgt

SEQ ID NO: 22 - DNA, 20 bp, synthetic construct
  source 1..20: mol_type = other DNA; note = TRAC_Site_3 gRNA
gagaatcaaaatcggtgaat

SEQ ID NO: 23 - DNA, 23 bp, synthetic construct
  source 1..23: mol_type = other DNA; note = TRAC_Site_3 NGS Forward Primer
gcctgggttggggcaaagaggga

SEQ ID NO: 24 - DNA, 23 bp, synthetic construct
  source 1..23: mol_type = other DNA; note = TRAC_Site_3 NGS Reverse Primer
gttgctccaggccacagcactgt

SEQ ID NO: 25 - DNA, 23 bp, synthetic construct
  source 1..23: mol_type = other DNA; note = OFF-TARGET SITE FOR HIST1H2B-GFP LOCUS
gctgtagcactccacgccgttgg

SEQ ID NO: 26 - DNA, 23 bp, synthetic construct
  source 1..23: mol_type = other DNA; note = OFF-TARGET SITE FOR HIST1H2B-GFP LOCUS
agcaaagcactgcacacagtggg

SEQ ID NO: 27 - DNA, 23 bp, synthetic construct
  source 1..23: mol_type = other DNA; note = OFF-TARGET SITE FOR HIST1H2B-GFP LOCUS
caggaagcactgcacactgtggg

SEQ ID NO: 28 - DNA, 23 bp, synthetic construct
  source 1..23: mol_type = other DNA; note = OFF-TARGET SITE FOR HIST1H2B-GFP LOCUS
aaagaagcactgcatgctgtagg

SEQ ID NO: 29 - DNA, 23 bp, synthetic construct
  source 1..23: mol_type = other DNA; note = OFF-TARGET SITE FOR HIST1H2B-GFP LOCUS
cctgcacccttgcacgccattgg

SEQ ID NO: 30 - DNA, 23 bp, synthetic construct
  source 1..23: mol_type = other DNA; note = OFF-TARGET SITE FOR HIST1H2B-GFP LOCUS
gaagaagcaccacacacagtagg

SEQ ID NO: 31 - DNA, 23 bp, synthetic construct
  source 1..23: mol_type = other DNA; note = OFF-TARGET SITE FOR HIST1H2B-GFP LOCUS
gaggaggcactgcacgccttggg

SEQ ID NO: 32 - DNA, 23 bp, synthetic construct
  source 1..23: mol_type = other DNA; note = OFF-TARGET SITE FOR HIST1H2B-GFP LOCUS
atagaagcacaacacgcagtggg

SEQ ID NO: 33 - DNA, 23 bp, synthetic construct
  source 1..23: mol_type = other DNA; note = OFF-TARGET SITE FOR HIST1H2B-GFP LOCUS
gctcaagcactgcaccccgtggg

SEQ ID NO: 34 - DNA, 23 bp, synthetic construct
  source 1..23: mol_type = other DNA; note = OFF-TARGET SITE FOR HIST1H2B-GFP LOCUS
agtgaagcaaaacacaccgtagg

SEQ ID NO: 35 - DNA, 23 bp, synthetic construct
  source 1..23: mol_type = other DNA; note = OFF-TARGET SITES FOR AAVS1 S10 LOCUS
agggacccagagagtggagaagg

SEQ ID NO: 36 - DNA, 23 bp, synthetic construct
  source 1..23: mol_type = other DNA; note = OFF-TARGET SITES FOR AAVS1 S10 LOCUS
gggatcccagccagtggagaagg

SEQ ID NO: 37 - DNA, 23 bp, synthetic construct
  source 1..23: mol_type = other DNA; note = OFF-TARGET SITES FOR AAVS1 S10 LOCUS
tggatcccagcgagtgaaggcgg

SEQ ID NO: 38 - DNA, 23 bp, synthetic construct
  source 1..23: mol_type = other DNA; note = OFF-TARGET SITES FOR AAVS1 S10 LOCUS
gggagcccagtcagtgaagaggg

SEQ ID NO: 39 - DNA, 23 bp, synthetic construct
  source 1..23: mol_type = other DNA; note = OFF-TARGET SITES FOR AAVS1 S10 LOCUS
aggagcccagagagagaagaggg

SEQ ID NO: 40 - DNA, 23 bp, synthetic construct
  source 1..23: mol_type = other DNA; note = OFF-TARGET SITES FOR AAVS1 S10 LOCUS
aagaacacagagagtgaagagag

SEQ ID NO: 41 - DNA, 23 bp, synthetic construct
  source 1..23: mol_type = other DNA; note = OFF-TARGET SITES FOR AAVS1 S10 LOCUS
gggaacccatggagtggagatgg

SEQ ID NO: 42 - DNA, 23 bp, synthetic construct
  source 1..23: mol_type = other DNA; note = OFF-TARGET SITES FOR AAVS1 S10 LOCUS
ggagacccagagggtgaagaggg

SEQ ID NO: 43 - DNA, 23 bp, synthetic construct
  source 1..23: mol_type = other DNA; note = OFF-TARGET SITES FOR AAVS1 S10 LOCUS
gggaacccagacagtgaaggggg

SEQ ID NO: 44 - DNA, 22 bp, synthetic construct
  source 1..22: mol_type = other DNA; note = OFF-TARGET SITES FOR AAVS1 S10 LOCUS
gagaacccaggagtgaagttgg

SEQ ID NO: 45 - DNA, 23 bp, synthetic construct
  source 1..23: mol_type = other DNA; note = OFF-TARGET SITES FOR AAVS1 S10 LOCUS
gagttagagcagaagaagaaagg

SEQ ID NO: 46 - DNA, 23 bp, synthetic construct
  source 1..23: mol_type = other DNA; note = OFF-TARGET SITES FOR AAVS1 S10 LOCUS
gagtctaagcagaagaagaagag

SEQ ID NO: 47 - DNA, 23 bp, synthetic construct
  source 1..23: mol_type = other DNA; note = OFF-TARGET SITES FOR AAVS1 S10 LOCUS
gaggccgagcagaagaaagacgg

SEQ ID NO: 48 - DNA, 23 bp, synthetic construct
  source 1..23: mol_type = other DNA; note = OFF-TARGET SITES FOR AAVS1 S10 LOCUS
gagtcctagcaggagaagaagag

SEQ ID NO: 49 - DNA, 23 bp, synthetic construct
  source 1..23: mol_type = other DNA; note = OFF-TARGET SITES FOR AAVS1 S10 LOCUS
aagtctgagcacaagaagaatgg

SEQ ID NO: 50 - DNA, 23 bp, synthetic construct
  source 1..23: mol_type = other DNA; note = OFF-TARGET SITES FOR AAVS1 S10 LOCUS
gagccggagcagaagaaggaggg

SEQ ID NO: 51 - DNA, 23 bp, synthetic construct
  source 1..23: mol_type = other DNA; note = OFF-TARGET SITES FOR AAVS1 S10 LOCUS
aagtccgaggagaggaagaaagg

SEQ ID NO: 52 - protein, 1368 aa, Streptococcus pyogenes [sequence omitted]

SEQ ID NO: 53 - protein, 1409 aa, Streptococcus thermophilus
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 54 - protein, 1082 aa, Neisseria meningitidis
MAAFKPNPINYILGLDIGIASVGWAMVEIDEEENPIRLIDLGVRVFERAEVPKTGDSLAMVRRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHISGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 55 - protein, 1395 aa, Treponema denticola
MKKEIKDYFLGLDVGTGSVGWAVTDTDYKLLKANRKDLWGMRCFETAETAEVRRLHRGARRRIERRKKRIKLLQELFSQEIAKTDEGFFQRMKESPFYAEDKTILQENTLFNDKDFADKTYHKAYPTINHLIKAWIENKVKPDPRLLYLACHNIIKKRGHFLFEGDFDSENQFDTSIQALFEYLREDMEVDIDADSQKVKEILKDSSLKNSEKQSRLNKILGLKPSDKQKKAITNLISGNKINFADLYDNPDLKDAEKNSISFSKDDFDALSDDLASILGDSFELLLKAKAVYNCSVLSKVIGDEQYLSFAKVKIYEKHKTDLTKLKNVIKKHFPKDYKKVFGYNKNEKNNNNYSGYVGVCKTKSKKLIINNSVNQEDFYKFLKTILSAKSEIKEVNDILTEIETGTFLPKQISKSNAEIPYQLRKMELEKILSNAEKHFSFLKQKDEKGLSHSEKIIMLLTFKIPYYIGPINDNHKKFFPDRCWVVKKEKSPSGKTTPWNFFDHIDKEKTAEAFITSRTNFCTYLVGESVLPKSSLLYSEYTVLNEINNLQIIIDGKNICDIKLKQKIYEDLFKKYKKITQKQISTFIKHEGICNKTDEVIILGIDKECTSSLKSYIELKNIFGKQVDEISTKNMLEEIIRWATIYDEGEGKTILKTKIKAEYGKYCSDEQIKKILNLKFSGWGRLSRKFLETVTSEMPGFSEPVNIITAMRETQNNLMELLSSEFTFTENIKKINSGFEDAEKQFSYDGLVKPLFLSPSVKKMLWQTLKLVKEISHITQAPPKKIFIEMAKGAELEPARTKTRLKILQDLYNNCKNDADAFSSEIKDLSGKIENEDNLRLRSDKLYLYYTQLGKCMYCGKPIEIGHVFDTSNYDIDHIYPQSKIKDDSISNRVLVCSSCNKNKEDKYPLKSEIQSKQRGFWNFLQRNNFISLEKLNRLTRATPISDDETAKFIARQLVETRQATKVAAKVLEKMFPETKIVYSKAETVSMFRNKFDIVKCREINDFHHAHDAYLNIVVGNVYNTKFTNNPWNFIKEKRDNPKIADTYNYYKVFDYDVKRNNITAWEKGKTIITVKDMLKRNTPIYTRQAACKKGELFNQTIMKKGLGQHPLKKEGPFSNISKYGGYNKVSAAYYTLIEYEEKGNKIRSLETIPLYLVKDIQKDQDVLKSYLTDLLGKKEFKILVPKIKINSLLKINGFPCHITGKTNDSFLLRPAVQFCCSNNEVLYFKKIIRFSEIRSQREKIGKTISPYEDLSFRSYIKENLWKKTKNDEIGEKEFYDLLQKKNLEIYDMLLTKHKDTIYKKRPNSATIDILVKGKEKFKSLIIENQFEVILEILKLFSATRNVSDLQHIGGSKYSGVAKIGNKISSLDNCILIYQSITGIFEKRIDLLKV